(19) United States
(12) Patent Application Publication
(10) Pub. No.: US 2013/0274210 A1
(43) Pub. Date: Oct. 17, 2013

(54) DENDRIMERS WITH A SACCHARIDE ENDING FOR ANTI-INFLAMMATORY PURPOSES

(71) Applicants: Jacques Prandi, Toulouse (FR); Germain Puzo, Auzeville Tolosane (FR); Cédric-Olivier Turrin, Toulouse (FR); Emilyne Blattes, Toulouse (FR); Alain Vercellone, Ramonville (FR); Jérôme Nigou, Auzeville Tolosane (FR); Jean-Pierre Majoral, Ramonville (FR); Anne-Marie Caminade, Toulouse (FR)

(72) Inventors: Jacques Prandi, Toulouse (FR); Germain Puzo, Auzeville Tolosane (FR); Cédric-Olivier Turrin, Toulouse (FR); Emilyne Blattes, Toulouse (FR); Alain Vercellone, Ramonville (FR); Jérôme Nigou, Auzeville Tolosane (FR); Jean-Pierre Majoral, Ramonville (FR); Anne-Marie Caminade, Toulouse (FR)

(73) Assignee: UNIVERSITE PAUL SABATIER TOULOUSE III, Toulouse (FR)

(21) Appl. No.: 13/819,872

(22) PCT Filed: Aug. 31, 2011
(86) PCT No.: PCT/FR11/52003

§ 371 Date: May 15, 2013

(30) Foreign Application Priority Data
Aug. 31, 2010 (FR) 1056902

(51) Int. Cl. C07H 15/207

(52) U.S. Cl. CPC C07H 15/207
USPC 514/25; 536/17.1

(57) ABSTRACT

The present invention relates to dendrimers with a monosaccharide, oligosaccharide or polysaccharide terminal group, to their preparation method and their therapeutic uses, notably anti-inflammatory uses.

### CROSS REFERENCE TO RELATED APPLICATIONS

The present application is the National Stage of International Application No. PCT/FR2011/052003 filed on Aug. 31, 2011, which published in French as WO 2012/089945 on Jul. 5, 2012. The PCT application claims priority to French Application No. 1056902 which was filed on Aug. 31, 2010. The above applications are incorporated herein by reference in their entirety.

### FIELD OF THE INVENTION

The present application relates to novel dendrimers with saccharide terminal groups, as well as to their preparation method and to their uses.

### BACKGROUND

Dendrimers are macromolecules consisting of monomers which are associated according to a tree-structured process around a multifunctional central core.

Dendrimers, also called “cascade molecules”, are highly branched functional polymers with a defined structure. These macromolecules are actually polymers since they are based on the association of recurrent units. However, dendrimers fundamentally differ from conventional polymers insofar that they have specific properties due to their tree-structured construction. The molecular weight and the shape of the dendrimers may be accurately controlled and the functions are located at the ending of the tree-structures, forming a surface, which makes them easily accessible.

Dendrimers are built step by step, by repeating a sequence of reactions allowing the multiplication of each recurrent unit and of the terminal functions. Each sequence of reactions forms what is called a “new generation”. The tree-structured construction is carried out by repeating a sequence of reactions with which a new generation and an increasing number of identical branches may be obtained at the end of each reaction cycle. After a few generations, the dendrimer assumes a highly branched and multifunctionalized globular shape by the numerous terminal functions present at the periphery.

Such polymers were notably described by Launay et al., Angew. Chem. Int. Ed. Engl., 1994, 33, 15/16, 1590-1592, or further Launay et al., Journal of Organometallic Chemistry, 1997, 529, 51-58.

Mannosylated lipoarabinomannan (or ManLAM) is a mycobacterial compound, stemming from Mycobacterium tuberculosis, capable, via the DC-SIGN receptor, of modulating the production of pro- and anti-inflammatory cytokines by human dendritic cells subject to stimulation by the lipopolysaccharide (LPS). The binding of ManLAM on the DC-SIGN receptor leads to decrease in the production of pro-inflammatory cytokines and to increase in the production of IL-10, an anti-inflammatory cytokine.

ManLAM is an amphiphilic macromolecule of about 18 kDa, the molecular structure of which comprises lipophilic domains on the one hand, and hydrophilic ends of the other hand, substituted with small α-(1→2) bound oligomannosides (mono-, di- or tri-mannosides), called caps. These caps are crucial for the observed biological activities of ManLAMs and their hydrolysis by an exo-mannosidase annihilates any biological activity of the ManLAM.

The amphiphilic structure of ManLAM leads to its organisation in an aqueous solution in the form of supramolecular aggregates (particulate ManLAM) which has the shape of a sphere with a diameter of about 30 nm. The lipophilic domains are sequestrated inside the particle which has at the surface the hydrophilic oligomannoside caps. After determination of the critical micellar concentration (CMC) of the ManLAM in solution, it was observed that it is only for values of the ManLAM concentration greater than CMC that the immunomodulating properties of ManLAM were observed, confirming that the biological active structure of the ManLAM is the particulate ManLAM.

Because of their structure, dendrimers have a strong density of terminal groups and therefore strong functional density at their periphery. It was therefore contemplated to mimic the supramolecular structure of the ManLAM with a dendrimer bearing at the surface the functions responsible for its biological activity, oligomannosides.

Mannosylated dendrimers have notably already been described in a review (Roy et al. Curr. Top. Med. Chem. 2008, 1237-1288) and in articles (Roy et al. J. Org. Chem. 2008, 73, 9292-9302; Wang et al. Proc. Natl. Acad. Sci. (USA) 2008, Vol 105, No. 10, 3690-3695; Rojo et al. Fed. Eur. Biochem. Soc. Lett. 2006, 580, 2402-2048), but none of these mannosylated dendrimers has an anti-inflammatory purpose.

Dendrimers having anti-inflammatory properties have also been described:
compounds of polyamidoamine (PAMAM) substituted at the surface with glucosamine (Shaunak et al. Nat. Biotechnol. 2004, 22, 977-984), or non-functionalized compounds (Chauhan et al. Biomacromolecules 2009, 10, 1195-1202),
a second generation phosphorus-containing dendrimer, the surface of which bears phosphonic acid functions (Fruchon et al. J. Leukoc. Biol. 2009, 85, 553-562).

However, no dendrimer with mannoside terminal groups and having an anti-inflammatory nature is described. The inventors from now on have developed a method giving the possibility of accessing this type of saccharide functionalization on dendrimers, with the purpose of mimicking the biological active particulate structure of ManLAM.

These dendrimers are excellent ligands of the DC-SIGN receptor and show immunomodulating properties in vitro, which makes them very good candidates for novel anti-inflammatory compounds.

### SUMMARY OF THE INVENTION

According to a first object, the present invention therefore relates to dendrimers with saccharide terminal groups at the ending of the final tree-structure.

According to a second object, the present invention also relates to the method for preparing such dendrimers.

According to another object, the present invention also relates to drugs comprising the dendrimers according to the invention.

According to another object, the present invention also relates to the use of dendrimers according to the invention for treating inflammatory diseases.

### BRIEF DESCRIPTION OF THE DRAWINGS

FIG. 1 represents EC50 (in nM) for the binding of the mannodendrimers to the DC-SIGN receptor expressed in HEK cells.

FIG. 2 illustrates the decrease of the TNF-α level secreted after co-incubation with LPS and mannodendrimers.

FIG. 3 illustrates the restoration of TNF-α production by AZN-D1, an antagonistic antibody of the DC-SIGN receptor with the mannodendrimer Gc3-TriM.

FIG. 4A illustrates the counting of the cells of broncho-alveolar washings of mice treated beforehand with forced feeding of mannodendrimer Gc3-TriM and nebulization of LPS.

FIG. 4B illustrates the counting of neutrophilic cells of broncho-alveolar washings of mice treated beforehand with forced feeding of mannodendrimer Gc3-TriM and nebulization of LPS.

### DETAILED DESCRIPTION

The present invention relates to dendrimers of generation g comprising:
a central core Φ of valency v;
generation chains with a tree-structure around the core:
an intermediate chain at the end of each chain of generation g or at the end of each bond around the core when g=0; and
a terminal group Σ at the end of each intermediate chain, characterized in that each group Σ, either identical or different, represents independently a monosaccharide, oligosaccharide or polysaccharide group, consisting of a saccharide units, and wherein:
g is an integer comprised between 0 and 10,
v is an integer comprised between 1 and 10,
σ is an integer comprised between 1 and 10,
said intermediate chain is represented by the formula (CI):

-A-(C═O)—Y—CH₂—O— (CI)

wherein:
Y represents a C₁-C₂₀ alkyl group, optionally substituted with a group selected from the group consisting of: halogen, OH, O-alkyl, CF₃, aryl, CN; preferably, Y represents a linear C₁-C₂₀ alkyl group,
A represents a bond or else a group:

—O—Ar—X—NR₁—

wherein:
Ar represents an aromatic ring optionally substituted with a group selected from the group consisting of: halogen, OH, O-alkyl, CF₃, aryl, CN, preferably a divalent group C₆H₄;
R₁ represents a hydrogen atom or a C₁-C₆ alkyl group, preferably a hydrogen atom, and
X represents a C₁-C₆ alkyl group, optionally substituted with a group selected from the group consisting of: halogen, OH, O-alkyl, CF₃, aryl, CN; preferably X represents a linear C₁-C₆ alkyl group.

Preferably, A represents a group —O—Ar—X—NR₁—, wherein Ar represents a divalent group —C₆H₄—, X represents a divalent group CH₂CH₂ and R₁ represents H.

Within the scope of the present description, by <<monosaccharide, oligosaccharide or polysaccharide group>> is meant a functional group selected from the group consisting of a monosaccharide, oligosaccharide and polysaccharide. By <<oligosaccharide>>, is meant a group consisting of 1 to 10 saccharide units. By <<polysaccharide>>, is meant a group consisting of more than 10 saccharide units. The bonds between the saccharide units are of the glycoside type and the links between the saccharide units are generally of the linear or branched type.

Within the scope of the present description, by <<saccharide unit>>, is meant the monomers selected from the group of monosaccharides, of molecular formula Cₓ(H₂O)ₓ. Monosaccharides are divided into trioses, tetroses, pentoses, hexoses and heptoses according to their number x of carbon atoms, as well as their derivative.

Mannose and glucose are examples of particularly advantageous monomers. Oligomannosides, polymannosides, oligoglucosides and polyglucosides are particularly interesting examples of a group Σ.

Within the scope of the present description, by <<glycoside bond>> is meant a chemical covalent bond between a saccharide unit and another group, typically another saccharide unit. Glycoside bonds are formed between the alcohol function of the hemiacetal carbon (or anomeric carbon) of a saccharide unit and the alcohol function of another organic compound, typically another saccharide unit. The glycoside bonds between saccharide units are of the α or β type, depending on the configuration of the saccharide units and are numbered according to the carbon atoms on either side of the oxygen atom connecting the saccharide units.

Examples of saccharide groups will be given in the description for illustrating these definitions.

Within the scope of the present invention, by <<dendrimer with saccharide terminal groups>> is meant a dendrimer bearing as terminal groups (or endings), monosaccharide, oligosaccharide or polysaccharide groups.

According to the present invention, the alkyl radicals represent saturated hydrocarbon radicals with a linear or branched chain, with 1 to 20 carbon atoms, preferably from 1 to 6 carbon atoms.

Notably mention may be made, when they are linear, of methyl, ethyl, propyl, butyl, pentyl, hexyl, octyl, nonyl, decyl, dodecyl, hexadecyl, and octadecyl radicals.

Notably, mention may be made, when they are branched or substituted with one or several alkyl radicals, of isopropyl, tert-butyl, 2-ethylhexyl, 2-methylbutyl, 2-methylpentyl, 1-methylpentyl and 3-methylheptyl radicals.

Among halogen atoms, mention will more particularly be made of fluorine, chlorine, bromine and iodine atoms, preferably fluorine.

Aryl designates a mono- or bi-cyclic hydrocarbon aromatic system with 6 to 10 carbon atoms.

Among aryl radicals, mention may notably be made of the phenyl or naphthyl radical more particularly substituted with at least one halogen atom.

Among alkylaryl radicals, mention may notably be made of the benzyl or phenethyl radical.

It will be appreciated that the compounds useful according to the present invention contain asymmetrical centers. These asymmetrical centers may be independently in an R or S configuration. It will appear to one skilled in the art that certain compounds useful according to the invention may also have geometrical isomerism. It should be understood that the present invention comprises individual geometrical isomers and stereoisomers and mixtures thereof, including racemic mixtures, of compounds described above. These isomers may be separated from their mixtures, by applying or adapting known methods, for example chromatography techniques or recrystallization techniques, or they are prepared separately from suitable isomers of their intermediates.

For the purposes of this text, it is understood that the tautomeric forms are comprised in the quoting of a given group, for example, a thio/mercampto or oxo/hydroxyl group.

Within the scope of the present invention, by <<with a tree-structure>> is meant the particular divergent structure of the chains of generations in generation layers around the central core Φ of valency v.

The different generation layers are obtained in successive generations by various methods of the divergent type (if one starts with the core) or convergent type (if one starts from one or several generation branches) in one or several steps. The dendrimers each generation, i.e. having a determined number of layers, may be isolated. The different generation layers (either internal or external) may be, like the core, organic, inorganic or consist of organic and inorganic elements.

The construction of these dendrimers may be strictly controlled. For example, in order to build a dendrimer, a series of generation branches is attached to the core and forms a first generation layer (generation 1) including at the periphery the same external functions and, by repeating the sequence of reactions used for building the first generation, a second generation layer is attached (generation 2) and then a third, a fourth, etc. . . .

The last generation layer (generation g) consists of chains of generation g. It comprises a plurality of identical chemical functions distributed at the periphery, each function forming or extending the free end of one of said generation branches of the last layer. At the end of these chains of generation g, intermediate chains are then grafted.

The number of these functions is an integer multiple, with a multiplying coefficient at least equal to 2, of the number of generation branches of said last generation layer.

Most often, the generation branches of all the internal layers of the dendrimer are identical and therefore form recurrent units.

The shape of the molecule generally has a spherical shape from generations 4 or 5. A dendrimer has a set of chemical functions and of locations with a same chemical environment allowing the grafting of one or several functional groups distributed at the periphery of the dendrimer.

Most often, the dendrimers of the invention include intermediate chains ending with a terminal group:
at the end of each generation chain, when g is greater than or equal to 1; or
at the end of each bond around the core, not connected to a generation chain, when g is equal to 0.

The dendrimers of the invention thus generally comprise v arms connected to the central core Φ, each of these arms being:
an arm of type (a), i.e. an arm formed with one or several generation chains with a tree-structure including at each of its ends, an intermediate chain ending with a terminal group Σ, when g is greater than or equal to 1; or
an arm of type (b), i.e. an arm formed with an intermediate chain ending with a terminal group Σ, when g is equal to 0.

According to a particular embodiment, the dendrimers exclusively include arms of type (a) bound to the central core Φ.

According to a particular embodiment, the dendrimers exclusively include arms of type (b) bound to the central core Φ.

Preferably, the dendrimers according to the invention correspond to commercial dendrimers to which intermediate chains and terminal groups have been grafted.

According to the invention, said commercial dendrimers are notably selected from dendrimers of the DAB-AM, PAMAM (notably Starbust®), PPI, polylysine and polytriazine (also called polymelamine) type, preferentially having terminal functions —NH₂, dendrimers of the PMMH (phenoxymethyl(methylhydrazone)) type, such as for example cyclotriphosphazene-PMMH, thiophosphoryl-PMMH or phosphorus-containing dendrimers such as:

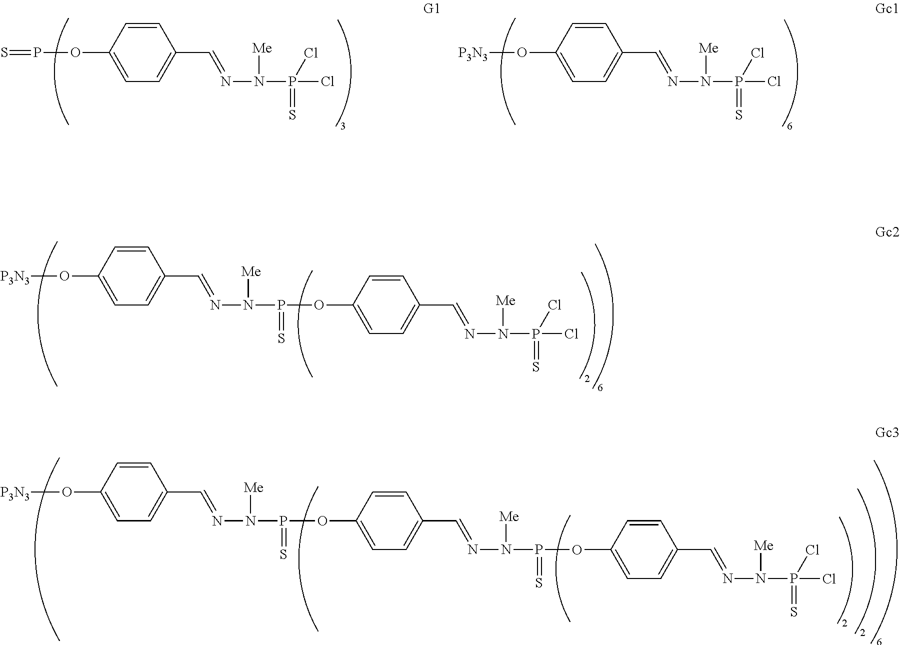

as well as subsequent generations.

All these dendrimers are marketed, some of them being available from Aldrich.

The present invention in particular relates to dendrimers such that the intermediate chain is represented by the formula (CI′):

—O—C₆H₄—CH₂—CH₂—NH—(C═O)—(CH₂)ₙ—CH₂—O— (CI′)
wherein n is an integer comprised between 1 and 12, and —C₆H₄— represents a divalent phenylene group.

Preferably, the generation chains are represented by the formula (CG):

—O—Ar′—Z═N—NR₂—(P═S)< (CG)
wherein:
   Ar′ is defined as Ar,
   Z is defined as X,
   R₂ is defined as R₁, and
   <represents two bonds located on the phosphorus atom.

Preferably, Ar′ represents a phenylene group, Z represents CH and R₂ represents a methyl.

Preferably, the generation chains are represented by the formula (CG′):

—O—C₆H₄(CH)═N—N(CH₃)—(P═S)< (CG′)

Preferably, the central core Φ is selected from the following groups:

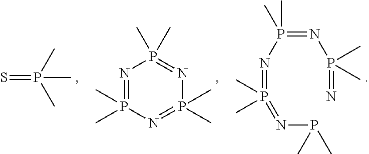

Preferably, the central core Φ is of formula:

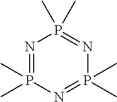

Preferably, v is an integer comprised between 1 and 8, preferably 3, 6 or 8.

Preferably, g is comprised between 0 and 10, more preferentially comprised between 0 and 4.

The number g represents the number of generations of the dendrimer. It is generally advantageous to adapt the number of generations according to the functionalization density and to the size of the desired dendrimer.

Preferably, σ is comprised between 1 and 3.

The terminal groups Σ thus consist independently of at most three saccharide units, either identical or different.

The terminal groups Σ are for example monosaccharides selected from trioses, tetroses, pentoses, hexoses or heptoses, or further di- or tri-saccharides, formed with different types of links between the saccharide units. These are typically di- or tri-saccharides bound in α-(1→6), α-(1→3) or branched in α-(1→6), α-(1→3).

Preferably, the saccharide units forming the terminal group Σ are selected from the group of hexoses.

Within the scope of the present invention, by <<hexose>> is meant a monosaccharide including six carbon atoms. They all have a carbonyl group: either an aldehyde function in position 1 (these are referred to aldohexoses), or a ketone function in position 2 (mainly), 3, 4, or 5 (these are referred to as ketone hexoses). Among the hexoses, mention may notably be made of ketohexoses (fructose, psicose, sorbose, tagatose), aldohexoses (allose, altrose, galactose, glucose, gulose, idose, mannose, talose) and deoxyose (fucose, fuculose, pneumose, quinovose, rhamnose).

The saccharide units forming the terminal group Σ are more particularly selected from the group of aldohexoses, i.e. allose, altrose, galactose, glucose, gulose, idose, mannose and talose.

According to an advantageous embodiment, the terminal groups Σ consist independently of the same saccharide unit.

According to another particularly advantageous embodiment, the terminal groups Σ at the end of the intermediate chains are identical.

According to a first aspect of this embodiment, the terminal groups Σ are identical and consist of mannose, preferably D-mannose, and are more preferentially dimannosides or trimannosides.

The terminal groups Σ are typically di- or tri-mannosides bound in α-(1→2), α-(1→3) or α-(1→6), or branched in α-(1→6), α-(1→3), as represented as an illustration in the following diagram:

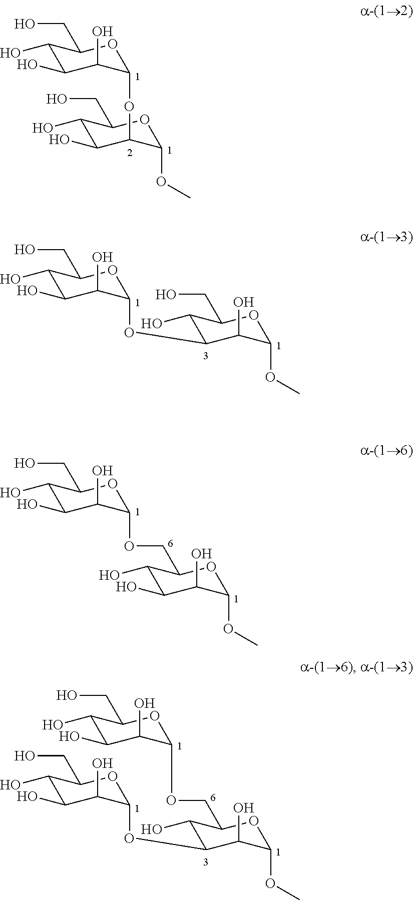 [SMILES: COC1OC(CO)C(O)C(O)C1OC1OC(CO)C(O)C(O)C1O ; COC1OC(CO)C(O)C(OC2OC(CO)C(O)C(O)C2O)C1O ; COC1OC(COC2OC(CO)C(O)C(O)C2O)C(O)C(O)C1O ; COC1OC(COC2OC(CO)C(O)C(O)C2O)C(O)C(OC2OC(CO)C(O)C(O)C2O)C1O]

According to a second aspect of this embodiment, the terminal groups Σ are identical and consist of glucose, preferably D-glucose, and are more preferentially diglucosides or triglucosides.

Among the polyglucosides, mention may notably be made of the derivatives of mycobacterial glucan, consisting of an α-(1→4) chain of α-D-glucopyranoside units which may be substituted in the position 6 with another α-D-glucopyranoside.

The terminal groups are typically di- or tri-glucosides bound in α-(1→6) or in α-(1→4), or branched in α-(1→4), α-(1→6), as represented as an illustration in the following diagram:

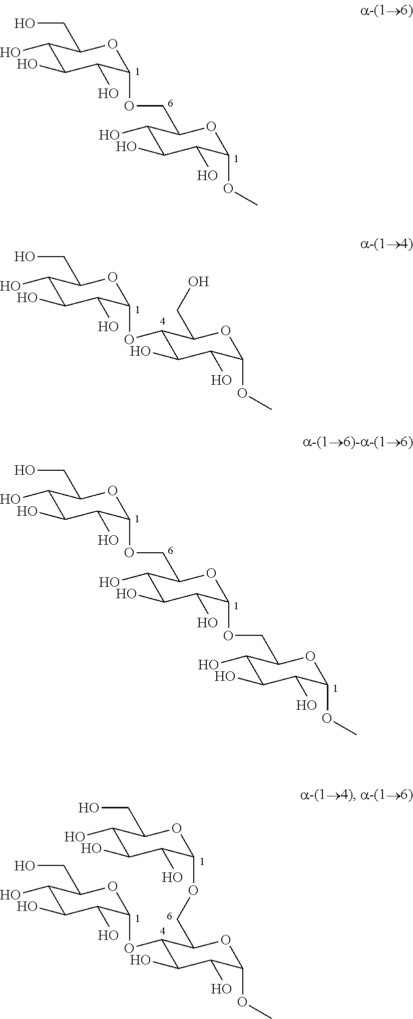 [SMILES: COC1OC(COC2OC(CO)C(O)C(O)C2O)C(O)C(O)C1O ; COC1OC(CO)C(OC2OC(CO)C(O)C(O)C2O)C(O)C1O ; COC1OC(COC2OC(COC3OC(CO)C(O)C(O)C3O)C(O)C(O)C2O)C(O)C(O)C1O ; COC1OC(COC2OC(CO)C(O)C(O)C2O)C(OC2OC(CO)C(O)C(O)C2O)C(O)C1O]

Preferably, the dendrimers according to the invention may be represented by the formula (1):

Φ-{{O—C₆H₄—(CH)═N—N(CH₃)—(P═S)<}^{g}[O—C₆H₄—CH₂CH₂—NH—(C═O)—(CH₂)ₙ—CH₂—O—Σ]₂}ᵥ (1)
wherein:

Φ, Σ, g, n, v and < are as defined earlier and { }^{g} designates the tree-structure of the generation chains of said dendrimer.

The radical —O—C₆H₄—CH₂—CH₂—NH— stems from tyramine.

Preferably, the dendrimers according to the invention have a diameter comprised between 1 and 100 nm, preferably less than 50 nm, and more preferentially less than 30 nm.

According to another object, the present invention also relates to the method for preparing the dendrimers mentioned above.

In the reaction described hereafter, it may be necessary to protect the reactive functional groups, for example, the hydroxy, amino, imino, thio, carboxy groups when they are desired in the final product, in order to avoid their undesirable participation in the reactions. Traditional protective groups may be used according to standard practices, for examples see T. W. Green and P. G. M. Wuts in Protective Groups in Organic Chemistry, John Wiley and Sons, 1991; J. F. W. McOmie in Protective Groups in Organic Chemistry, Plenum Press, 1973.

According to the invention, a first embodiment of the method for preparing a dendrimer according to the invention comprises the reaction of the dendrimer of generation g comprising:
a central core Φ of valency v;
tree-structured generation chains around the core;
—NHR₁ terminal groups;
   with an azyl-azide compound of formula (3):

N₃—(C═O)—Y—CH₂—O—Σ (3)

wherein: g, Φ, v, R₁, Y and Σ are as defined earlier and wherein the —NHR₁ groups are optionally in the form of ammonium ions —NH₂R₁⁺, in equilibrium with the conjugate base of a weak or strong acid.

According to the invention an advantageous embodiment of the method for preparing a dendrimer according to the invention comprises the reaction of the dendrimer of formula (2):

Φ—{{O—Ar′—Z═N—NR₂—(P═S)<}^{g}[O—Ar—X—NHR₁]₂}ᵥ (2)

with an acyl-azide compound of formula (3):

N₃—(C═O)—Y—CH₂—O—Z (3)

wherein: Φ, Ar, Ar′, X, Y, Z, R₁, R₂, Σ, g, v, < and { }^{g} are as defined earlier and wherein the —NHR₁ groups are optionally in the form of ammonium ions NH₂R₁⁺, in equilibrium with the conjugate base of a weak or strong acid.

More specifically, according to this reaction, the —NHR₁ groups of the dendrimer of formula (2) are acylated with an acyl-azide compound of formula (3).

According to the invention, the compound of formula (2) may be obtained by coupling between a dendrimer of formula (4):

Φ—{{O—Ar′—Z═N—NR₂—(P═S)<Cl₂}^{g}}ᵥ (4)

and a compound of formula (5):

HO—Ar—X—NR₁-PG1 (5)

wherein PG1 represents a protective group, typically a Boc group, followed by deprotection of the protective groups PG1 of the thereby formed dendrimer of formula (6):

Φ—{{O—Ar′—Z═N—NR₂—(P═S)<}^{g}[O—Ar—X—NR₁—PG1]₂}ᵥ (6)

More specifically, according to the coupling reaction, the chlorine atoms of the function (P═S)<Cl₂ of the dendrimer of formula (4) are substituted with the oxygen atom of the alcohol function of the compound of formula (5).

More specifically, according to the deprotection reaction, the protective groups PG1 of the dendrimer of formula (6) are removed in the presence of an acid, such as trifluoroacetic acid, in order to obtain a dendrimer including amino groups —NHR₁ as surface functions. The groups NHR₁ of this dendrimer are possibly obtained as ammonium ions NH₂R₁⁺, in equilibrium with the conjugate base of a strong acid, such as CF₃COO⁻.

According to an advantageous embodiment, the method for preparing a dendrimer of formula (1) according to the invention comprises:
(i) the coupling reaction between the corresponding dendrimer having a terminal function —(P═S)<Cl₂ and tyramine, the nitrogen atom of which is protected by a protective group PG1 (N-PG1 tyramine), typically a Boc group,
(ii) followed by the deprotection reaction of the protective groups PG1 of the dendrimer obtained in (i),
(iii) followed by the reaction of the dendrimer obtained in (ii) with an acyl-azide compound of formula (3′):

N₃(C═O)—(CH₂)ₙ—CH₂—O—Σ (3′)

wherein n and Σ are as defined earlier.

According to the invention, by <<corresponding dendrimer>> is meant the dendrimer of the same generation having the same cores, generation chains, intermediate chains and distinct terminal groups.

The starting corresponding dendrimers are commercially available (Aldrich) or may be prepared according to methods known per se. These dendrimers include surface functions —(P═S)<Cl₂ and are preferably selected from:

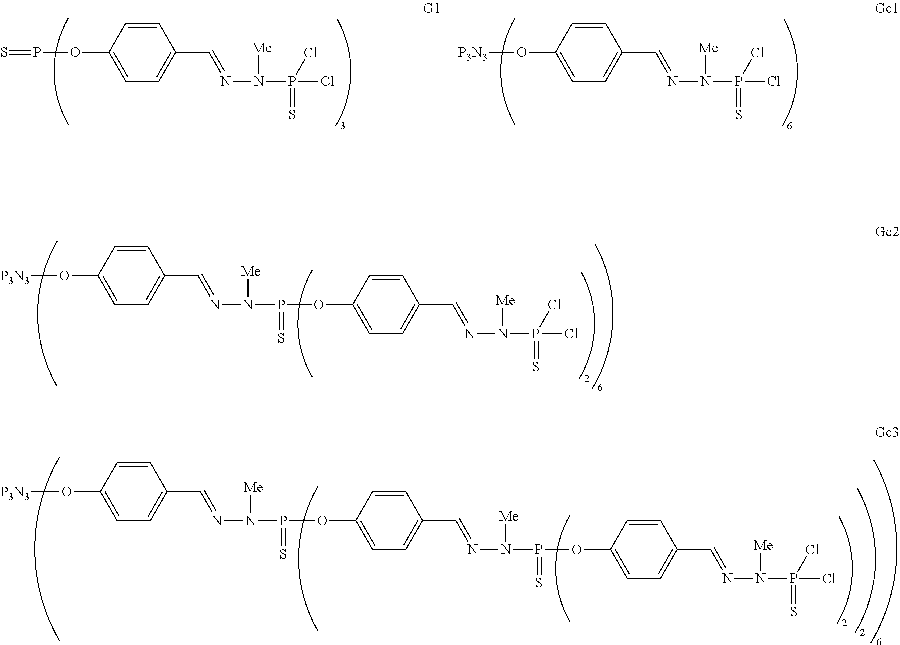

The coupling may be achieved by applying or adapting the method described in Tet. Lett. 2009, 50, 2078.

Deprotection may be achieved by applying or adapting the method described in Tet. Lett. 2009, 50, 2078.

More specifically, according to step (i), the chlorine atoms of the functions —(P═S)<Cl₂ are substituted with the oxygen atom of the phenol function of the protected tyramine, N-PG1 tyramine, preferably in THF at room temperature and in the presence of an inorganic base, typically an alkaline metal carbonate, for example, Cs₂CO₃. Typically, step (i) is carried out by using 1.1 molar equivalents of N-PG1 tyramine and 2.2 basic molar equivalents per chlorine atom to be substituted. This reaction may be conducted at a temperature comprised between −60° C. and 65° C.

More specifically, according to step (ii), the protective groups PG1 of the dendrimer obtained according to step (i), via coupling with N-PG1 tyramine, are removed typically in an acid medium, in order to obtain a dendrimer including —NH₂ groups as surface functions. Typically, step (ii) is carried out in a (3:1) mixture of dichloromethane and trifluoroacetic acid. The —NH₂ groups of this dendrimer are then possibly obtained as ammonium salts, typically NH₃⁺CF₃COO⁻.

More specifically, according to step (iii), the —NH₂ groups of the obtained dendrimer according to step (ii) are acylated with an acyl azide compound of formula (3′). Typically, step (iii) is carried out in an aqueous buffer and 3 to 4 molar equivalents of acyl-azide compound of formula (3′) per —NH₂ to be acylated are generally used.

Optionally, said method may also comprise the step consisting of isolating the product obtained at the end of steps (i), (ii) and/or (iii).

As an illustration, the method according to the invention may be carried out by applying the following reaction scheme:

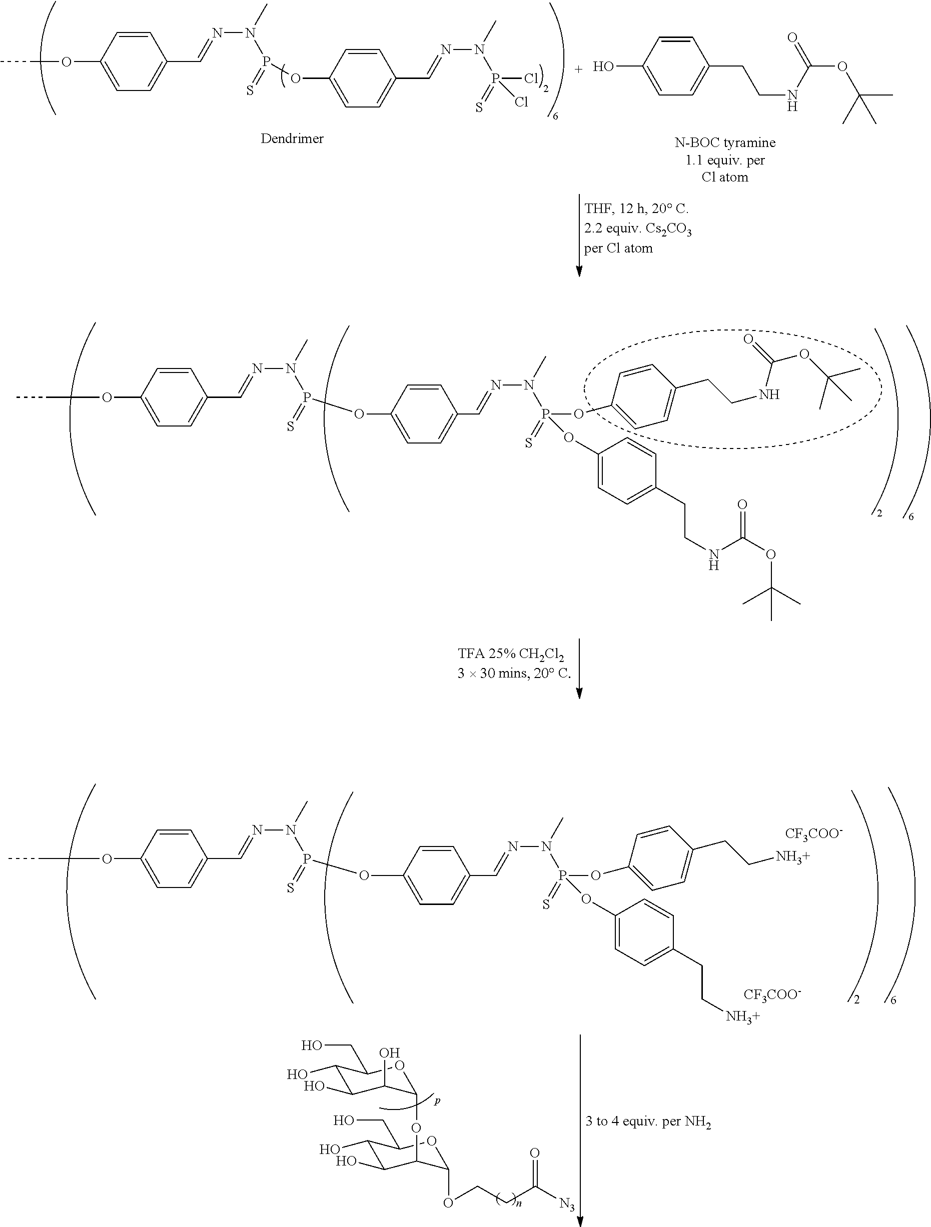

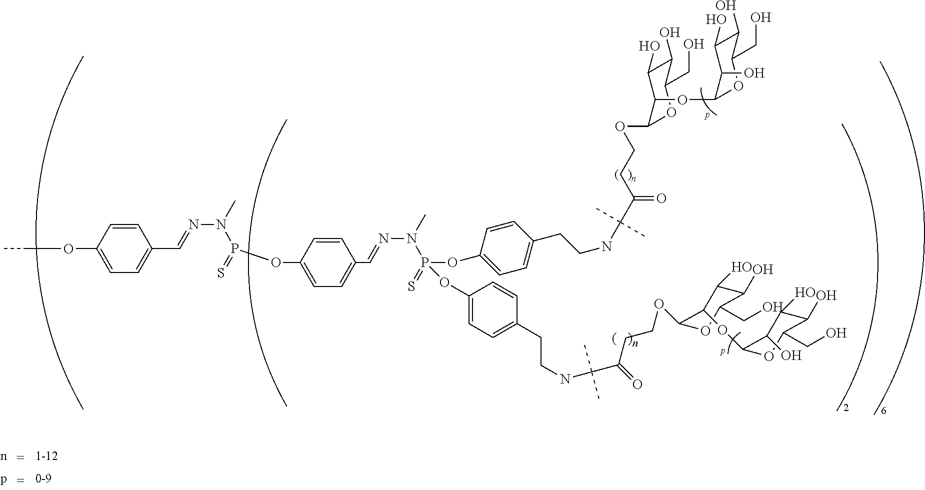

The compounds of formula (3) are therefore also part of the present invention.

The present invention also relates to compounds of formula (3):

N₃—(C═O)—Y—CH₂—O—Σ (3)
wherein Σ and Y are as defined earlier.

Preferably, the compounds of formula (3) fit the formula (3′):

N₃—(C═O)—(CH₂)ₙ—CH₂—O—Σ (3′)

wherein:
is as defined earlier.
n is an integer comprised between 1 and 12.

Preferably, in formulae (3) and (3′), the group Σ is formed with at most three saccharide units, either identical or different (σ is comprised between 1 and 3).

The present invention in particular relates to the following compounds:

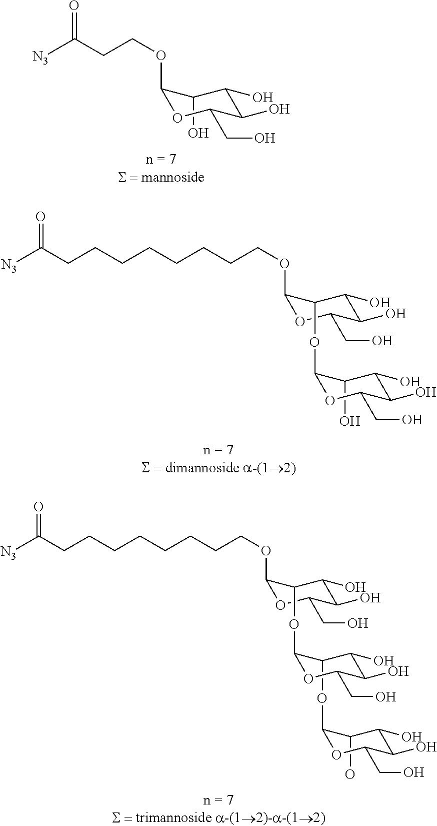 [SMILES: [N-]=[N+]=NC(=O)CCOC1OC(CO)C(O)C(O)C1O ; [N-]=[N+]=NC(=O)CCCCCCCCOC1OC(CO)C(O)C(O)C1OC1OC(CO)C(O)C(O)C1O ; [N-]=[N+]=NC(=O)CCCCCCCCOC1OC(CO)C(O)C(O)C1OC1OC(CO)C(O)C(O)C1OC1OC(CO)C(O)C(O)C1O]

The compounds of formula (3) may be obtained from esters of formula (7) according to the method comprising:
(i′) the reaction of the ester of formula (7):

R₃O—(C═O)—Y—CH₂—O—Σ (7)
with hydrazine hydrate,
(ii′) followed by the reaction of the compound obtained in (i′) with sodium nitrite in acid medium,
   wherein Σ and Y are as defined earlier and R₃ represents a linear C₁-C₆ alkyl chain, preferably a methyl.

More specifically, according to step (i′), an ester of formula (7) is treated with an excess of hydrazine hydrate, typically in ethanol, in order to obtain the corresponding hydrazide.

More specifically, according to step (ii′), the hydrazide obtained according to step (i′) is treated with sodium nitrite in an acid medium, typically in the presence of an acid solution in dioxane, such as a hydrochloric acid solution in dioxane in order to obtain an acyl-azide of formula (3).

Preferably the group R₃ represents a methyl.

In particular, the compounds of formula (3′) may be obtained from esters of formula (7′) according to the method comprising:
(i″) the reaction of the ester of formula (7′):

R₃O—(C═O)—(CH₂)ₙ—CH₂—O—Σ (7′)
with hydrazine hydrate,
(ii″) followed by the reaction of the compound obtained in (i″) with sodium nitride in an acid medium,

wherein n, Σ and R₃ are as defined earlier.

As an illustration, the compounds of formula (3) may be obtained by applying the following reaction scheme:

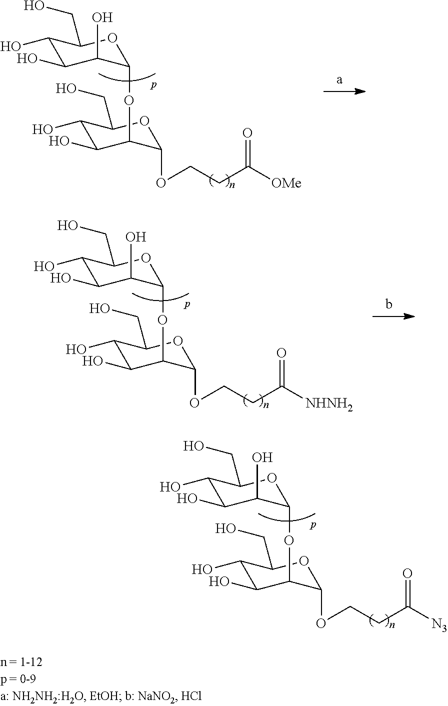

The esters of formula (7) may be obtained by applying or adapting methods known per se and/or within the reach of one skilled in the art, notably those described by Larock in Comprehensive Organic Transformations, VCH Pub., 1989, or by applying or adapting the methods described in the following examples.

The esters of formula (7) may notably be obtained from compounds of formula (8):

Sac(LG)(OPG2)_{y}(OPG3)_{z} (8)

wherein:
Sac represents the carbon backbone corresponding to a saccharide unit selected from trioses, tetroses, pentoses, hexoses and heptoses,
LG represents a leaving group, typically a thioether, located on carbon no. 1 of the carbon backbone Sac,
PG2 and PG3 represent different protective groups and such that, under the conditions of deprotection of PG2, the group PG3 is not deprotected, and vice versa,
y represents an integer comprised between 0 and 5 and z represents an integer comprised between 1 and 6; y and z are such that all the other alcohol functions of the compounds of formula (8) are protected, either with a PG2 group, or with a PG3 group.

Thus, the compounds of formula (7) may be obtained from compounds of formula (8), according to the method comprising:
(i′″) the reaction of a compound of formula (8) with the ester of formula (9):

R₃O—(C═O)—Y—CH₂—OH (9)
(ii′″) optionally, the reaction for deprotection of the protective groups PG2 and the reaction of the deprotected product obtained with a compound of formula (8), this sequence being repeated as many times as required for obtaining the number a of saccharide units,
(ii′″) the reaction for deprotection of all the protective groups (PG2 and PG3) in order to obtain the compound of formula (7):

R₃O—(C═O)—Y—CH₂—O—Σ (7)
wherein Y and Σ are as defined earlier and R₃ represents a linear C₁-C₆ alkyl.

When y is equal to 0, i.e. when no alcohol function is protected by a PG2 group, the saccharide group Σ is of the monosaccharide type.

When y is equal to 1, i.e. when a single alcohol function is protected by a PG2 group, the obtained saccharide group Σ is of the linear type.

When y is strictly greater than 1, i.e. when several alcohol functions are protected with a PG2 group, the obtained saccharide group Σ is of the branched type.

More specifically, according to step (i′″), the leaving group LG of the compound of formula (8) is substituted with the oxygen atom of the alcohol function of the compound (9) (glycosylation reaction). This reaction is preferably conducted in anhydrous dichloromethane and in the presence of N-iodosuccinimide (NIS) and of a catalytic amount of trimethylsilyl trifluoromethanesulfonate (TMSOTf).

More specifically, according to step (ii′″), the PG2 group(s) is(are) deprotected and the released alcohol function(s) then react(s) with the compound of formula (8) under the same conditions as in step (i′″), by forming glycoside bonds. This step is optionally repeated and this as many times as required for obtaining the number a of saccharide units intended for the group Σ.

More specifically, according to step (iii′″), the PG3 groups are finally deprotected, as well as the possible PG2 groups, and the compound of formula (7) is obtained, the alcohol functions of which are free.

The PG2 group is typically an acetate, which may be removed with a sodium methanolate solution in methanol.

The PG3 group is typically a benzyl ether, which may be removed by hydrogenolysis in the presence of a catalytic amount of palladium on coal.

The saccharide derivatives of formula (8) may be obtained according to methods described in literature and by using current knowledge of sugar chemistry. Thus, they may notably be synthesized by applying or adapting the article of Peters et al. Liebigs Ann. Chem. 1991, 135-141 which describes the synthesis from D-mannose of a mannoside including in position 1 a thioethyl group, in position 2 an acetate group and in position 3, 4 and 6 benzyl ethers, as illustrated in the following scheme:

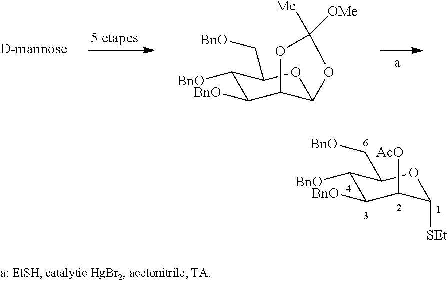

Peters et al. also describes the coupling of this thiomannoside and the synthesis of a α-(1→2) dimannoside.

As an illustration, the method according to the invention may be carried out by applying the following reaction scheme;

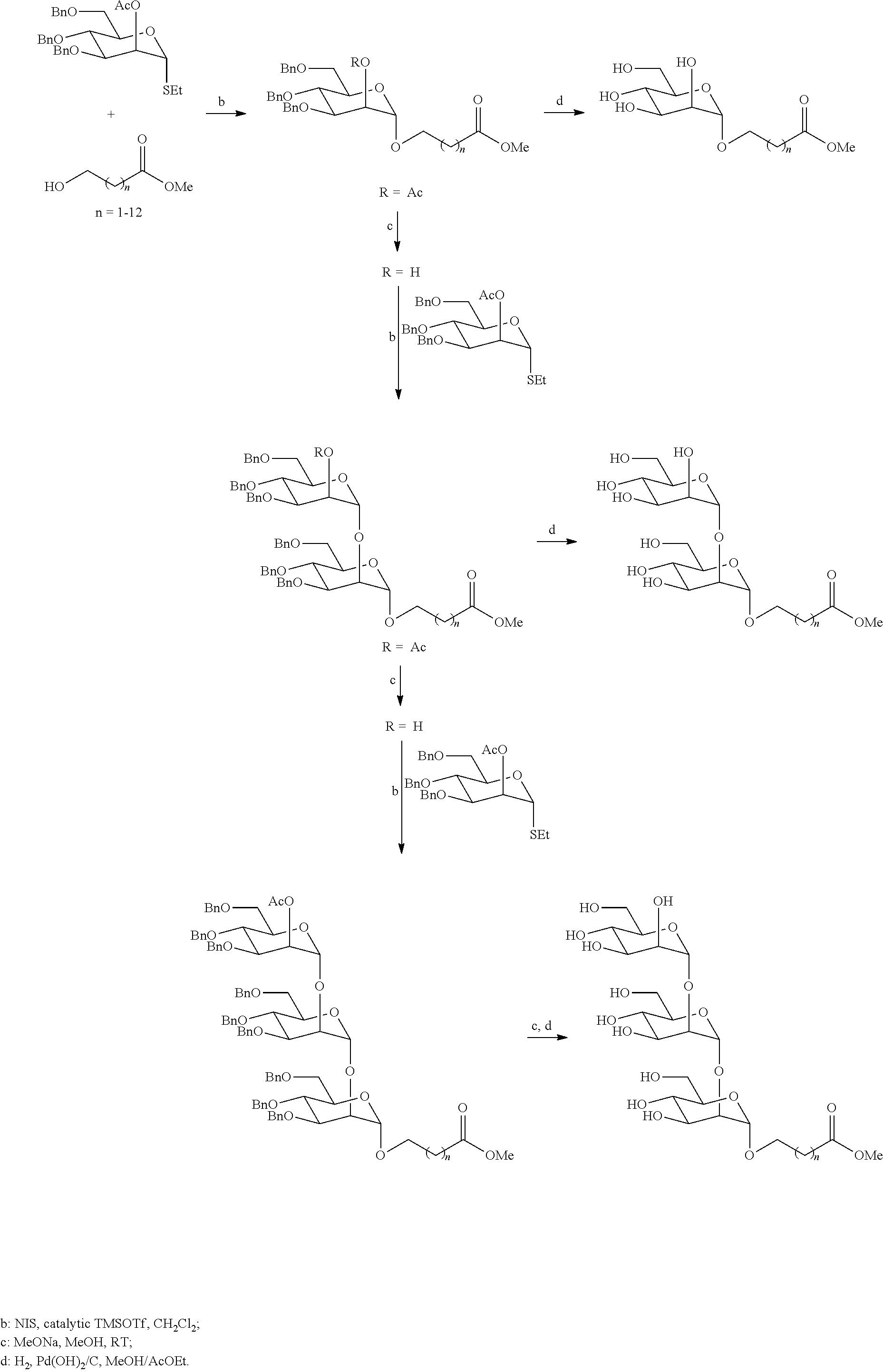

The thereby prepared compounds may be recovered from the mixture of the reaction by traditional means. For example, the compounds may be recovered by distilling the solvent of the mixture of the reaction or if necessary after distillation of the solvent of the mixture of the solution by pouring the remainder in water followed by extraction with an organic solvent immiscible with water, and by distilling the solvent of the extract. Further, the product may, if this is desired, be further purified with various techniques, such as recrystallization, reprecipitation and various chromatography techniques, notably column chromatography or preparative thin layer chromatography.

The compounds according to the present invention may be easily prepared or formed during the process of the invention, as solvates (for example hydrates). The hydrates of the compounds useful according to the present invention may be easily prepared by recrystallization of a mixture of aqueous/organic solvent, by using organic solvents such as dioxane, tetrahydrofurane or methanol.

The basic products or the intermediates are commercially available and/or may be prepared by applying or adapting known methods, for example, methods such as those described in the reference examples.

The inventors have discovered that the modified dendrimers according to the invention have particularly advantageous properties, notably anti-inflammatory properties.

According to another object, the present invention also relates to a drug comprising one of the dendrimers mentioned above and a pharmaceutically acceptable excipient.

The pharmaceutical compositions according to the invention may be presented in forms intended for administration via a parenteral, oral, rectal, permucosal or percutaneous route, by applying or adapting formulations generally used.

They will be therefore appear as solutes or injectable suspensions or multidose flasks, in the form of exposed or coated tablets, dragees, capsules, gelatin capsules, pills, cachets, powders, suppositories or rectal capsules, solutions or suspensions for percutaneous use in a polar solvent, for permucosal use.

The excipients which are suitable for such administrations are derivatives of cellulose or microcrystalline cellulose, earth alkaline carbonates, magnesium phosphate, starches, modified starches, lactose for solid forms.

For rectal use, cocoa butter or polyethylene glycol stearates are the preferred excipients.

For parenteral use, water, aqueous solutes, saline, isotonic solutes are the carriers which are the most conveniently used.

The dosage may vary within wide limits, (0.5 mg to 1000 mg) depending on the therapeutic indication and on the administration route, as well as on the age and weight of the subject.

The present invention particularly relates to the dendrimers as described earlier for their use for treating and/or preventing inflammatory disorders.

The inventors have actually shown that the dendrimers according to the invention have the capability of lowering the level of secreted inflammatory cytokines (TNF-α) by human dendritic cells stimulated with LPS and of thereby inhibiting stimulation by LPS.

The present invention more particularly relates to the use of the dendrimers mentioned above for which said dendrimer has affinity for the DC-SIGN receptor in its membrane form.

The inventors have actually shown that the dendrimers according to the invention have better affinity for ManLAM than for DC-SIGN in the membrane form.

### EXAMPLES

The present invention will be better understood upon reading the following examples.

I. General Methods

1. α-Glycosylation with Thiomannoside 2

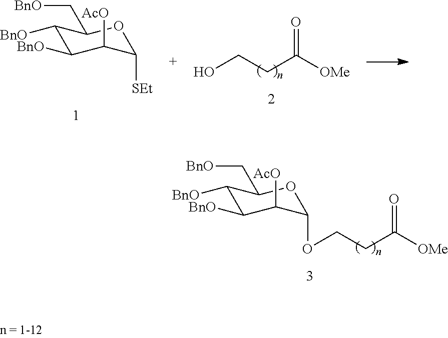

1.0 equivalent of compound 1 and 1.1 equivalents of alcohol to be glycosylated (either the compounds 2 or the hydroxyl in the position 2 of the mannose during synthesis) are dissolved in anhydrous dichloromethane (0.4M) in the presence of an activated 3 Å molecular sieve. The reaction medium, under an inert atmosphere is cooled in ice. Once it has reached the temperature, 1.3 equivalents of N-iodosuccinimide are added. After 20 minutes of stirring, 0.08 equivalent of a molar solution of trimethylsilyl trifluoromethanesulfonate in dichloromethane are added and the reaction is left at 0° C. for 2 hours. The reaction medium is neutralized with a few drops of triethylamine. After filtration on Celite 545, the medium is hydrolyzed by a saturated sodium thiosulfate solution. The aqueous phase is extracted twice with dichloromethane. The organic phase is dried on magnesium sulfate, filtered and then evaporated. A purification with a chromatographic column on silica gel with ethyl acetate/petroleum ether (1:3) gives the possibility of obtaining the intended product 3 either separated or not from the isomer 3.

2. Deacytelation

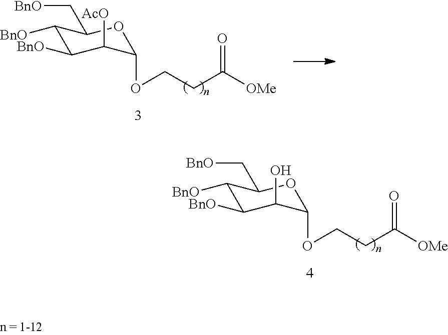

The compound 3 to be deacytelated is dissolved in anhydrous methanol (at 0.08 M) under an inert atmosphere. A small piece of solid sodium is added thereby forming sodium methanolate in situ. The reaction medium is stirred for one night at room temperature. At the end of the reaction, the reaction medium is neutralized with proton-exchange resin (tracked with pH paper, pH 7), filtered and evaporated. With purification by a chromatographic column on silica gel with toluene/ethyl acetate (5:1), it is possible to obtain the pure compound 4.

The compound 4 may then be reacted with a compound of the type of the compound 1, in order to obtain, by forming a new glycoside bond, a compound of the disaccharide type.

3. Debenzylation by Catalytic Hydrogenation

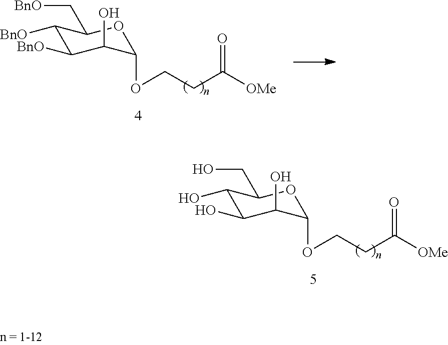

The compound to be debenzylated 4 is dissolved in methanol (0.08 M) in the presence of a catalytic amount of palladium hydroxide on activated coal, in an atmosphere saturated with dihydrogen. A control TLC gives the possibility of checking the end of the reaction. The reaction medium is then filtered on celite 545 and then evaporated. No purification step on a chromatographic column is required. The pure debenzylated compound 5 is obtained.

4. Hydrazidation of the Methyl Ester Function

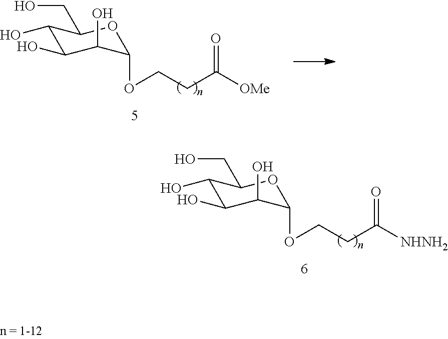

The thereby deprotected compound 5 is treated at room temperature for one night with 53.0 equivalents of hydrazine hydrate dissolved in absolute ethanol (125.0 equivalents). A control TLC (AE/MeOH/H₂O) (8:3:1) gives the possibility of checking the disappearance of the initial compound. The reaction medium is then evaporated and then freeze-dried several times with water. The obtained product 6 does not undergo any purification step and is directly engaged into the reaction for substitution of the dendrimers with an ammonium terminal group.

5. Attaching the Tyramine onto the Gcₙ Dendrimers

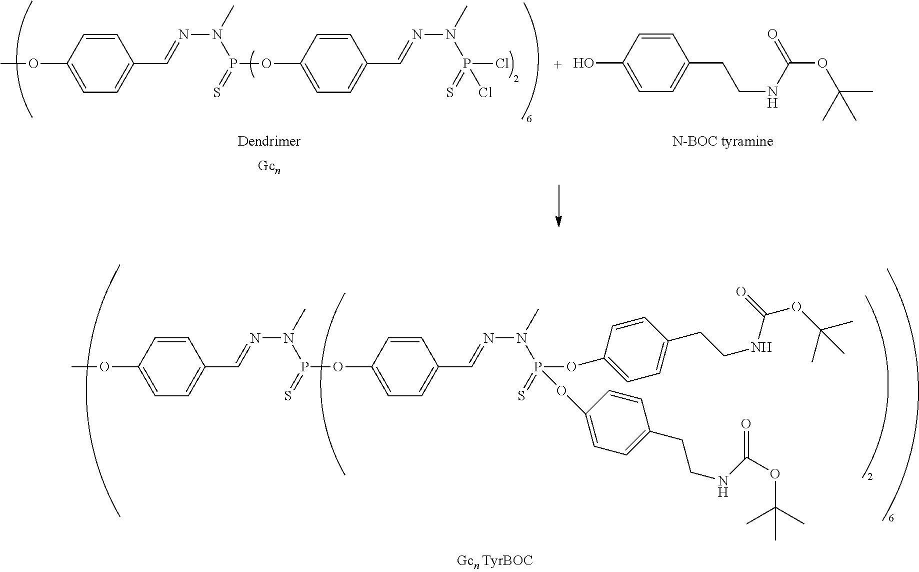

This coupling may be achieved by applying or adapting the method described in Tet. Lett. 2009, 50, 2078.

1.0 equivalent of Gcₙ dendrimer is dissolved in anhydrous THF at the concentration of 1 to 2 mM in the presence of 7.5×2ⁿ equivalents of solid cesium carbonate. 6.3×2ⁿ equivalents of N-BOC-tyramine are added subsequently and the reaction medium is stirred for 12 hours at room temperature. The reaction is tracked with phosphorus (³¹P) NMR of the crude reaction medium (reference by means of a C₆D₆ capillary). At the end of the reaction, the reaction medium is centrifuged for 10 minutes at 10,000 g at room temperature (rotor 25.50, Jouan), and the supernatant is then recovered and evaporated. With purification by a chromatographic column on silica gel, it is possible to obtain the pure GcₙTyrBOC compound, with ethyl acetate/petroleum ether (1:2 to 1:1).

6. Deprotection of the BOC group of the GcₙTyrBOC

The deprotection may be achieved by applying or adapting the method described in Tet. Lett. 2009, 50, 2078.

(a) Preparation of the Dendrimers with an Ammonium Terminal Grow

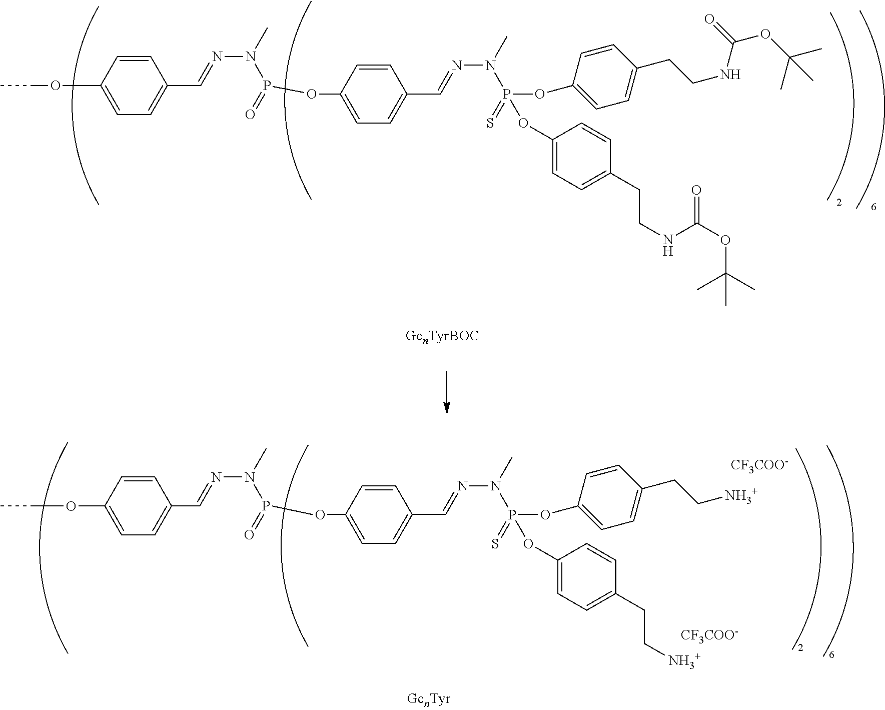

The GcₙTyrBOC dendrimer to be deprotected is taken up in 5 mL of dichloromethane (2 mM) with 25% of trifluoroacetic acid (Sigma) and is stirred for 30 mins at room temperature. The reaction medium is then dry evaporated. The compound undergoes at least three times this step and this until complete deprotection. With 3 co-evaporations with methanol, it is possible subsequently to remove the excess trifluoroacetic acid (tracked with ¹⁹F NMR). The GcₙTyr dendrimer soluble in water is then freeze-dried and kept at −20° C. under an inert atmosphere.

7. Coupling the Oligomannosides onto the GcₙTyr Dendrimer with an Ammonium Terminal Group

(a) Formation of the Acyl-Azide:

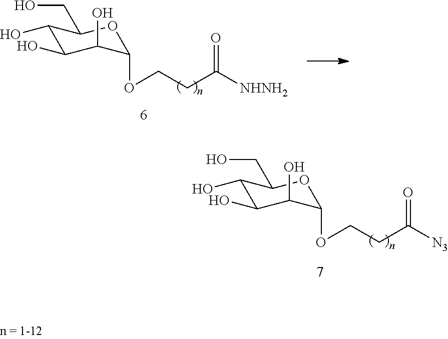

The compound 6 (6×2ⁿ equivalents wherein n is the generation of the Gcₙ dendrimer to be substituted) is dissolved at a concentration of 6.10⁻² M in anhydrous DMF. The solution is cooled down to −25/−30° C., and 36×2ⁿ equivalents of HCl (4M solution in dioxane) are added, followed by 12×2ⁿ equivalents of solid sodium nitrite (NaNO₂). The mixture is stirred for 30 minutes at −25/−30° C., and then with a control TLC (AE/MeOH/H₂O (8:3:1)), it is possible to check the disappearance of the initial hydrazide and the formation of a new less polar compound 7 (RF=0.7 for the monomannoside). 9×2ⁿ equivalents of sulfamic acid (70 mg·mL⁻¹ solution in DMF) are added, the reaction is stirred for 15 minutes at −25/−30° C. and the obtained solution of compound 7 is used as such for the next step.

(b) Substitution of the Dendrimers:

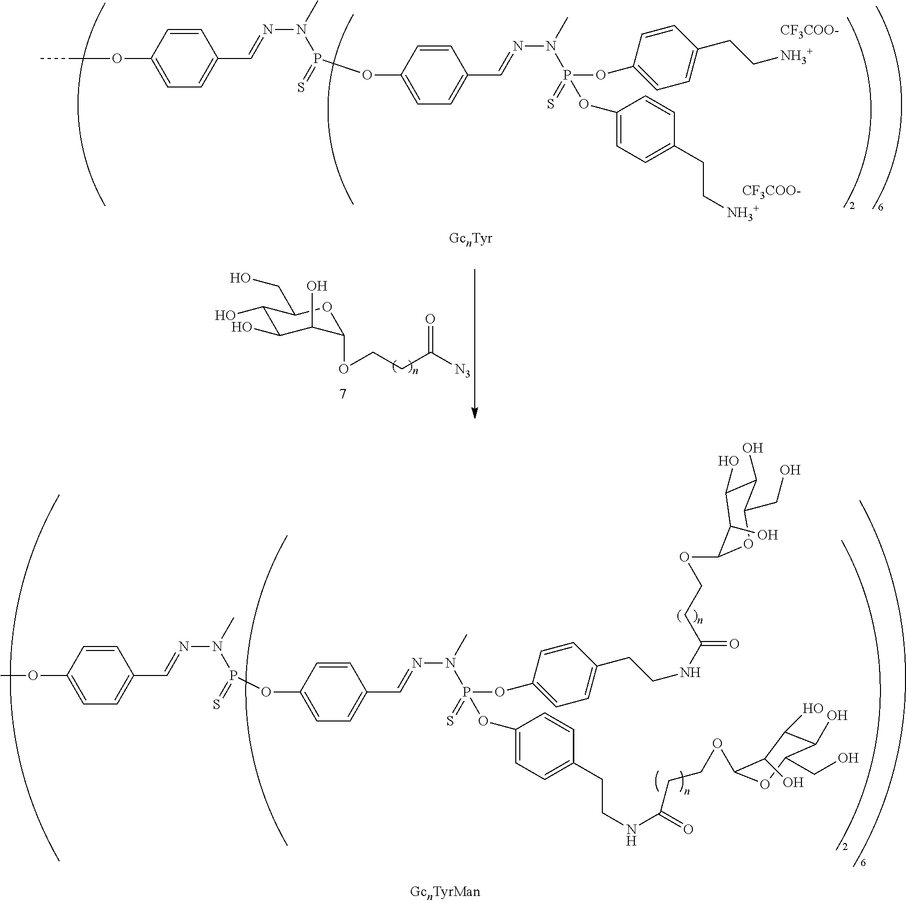

To the previous reaction medium is added the dendrimer with an ammonium terminal group (GcₙTyr, prepared in step 6) at a concentration of 1.10⁻² M in anhydrous DMF. The pH of the mixture is adjusted by adding triethylamine dropwise until a basic pH is obtained (pH≧9). The whole is stirred at 4° C. for 6 hours.

After 6 hours, the reaction medium is again treated with 6×2ⁿ equivalents of a solution of acyl azide mannoside at 4° C. This step is repeated as many times as required in order to obtain total substitution of the dendrimer.

(c) Purification of the Mannosyl Dendrimer:

The reaction mixture is evaporated and the residue is taken up in a minimum volume of H₂O in order to be deposited on a size exclusion chromatography column (fine Biogel P-2, 60 mL, 0.16 mL·min⁻¹). Each fraction is analyzed on TLC (AE/MeOH/H₂O (8:3:1)) and those containing the pure mannodendrimer GcₙTyrMan (RF=0, development with UV and H₂SO₄ 5% EtOH) are freeze-dried forming a yellow-orangey solid.

II. Prepared Molecules

The synthesis route of the intermediates described above allowed the following molecules to be obtained:

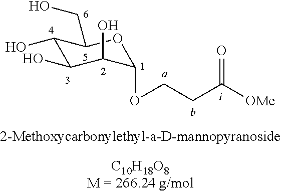 [SMILES: COC(=O)CCOC1OC(CO)C(O)C(O)C1O]

[α]_{D}²⁵=+55 (c=1.0, MeOH)

Aspect: colorless oil

¹H NMR data: (250 MHz, CD₃OD, 25° C.) δ=4.74 (1H, d, ³J₁₋₂=1.5 Hz, H-1), 4.02 (1H, td, ²Jₐ₋ₐ=10.0 Hz, ³J_{a-b}=6 Hz, H-a), 3.86 (1H, dd, ²J₆₋₆=12.0 Hz, ³J₅₋₆=2.5 Hz, H-6), 3.78 (1H, dd, ³J₂₋₃=2.5 Hz, ³J₁₋₂=1.5 Hz, H-2), 3.74 (1H, dd, ²J₆₋₆=12.0 Hz, ³J₅₋₆=5.0 Hz, H-6), 3.71 (1H, td, ²Jₐ₋ₐ=10 Hz, ³J_{a-b}=6 Hz, H-a), 3.71 (3H, s, H-OMe), 3.67-3.60 (2H, m, H-3 and H-4), 3.55 (1H, ddd, ³J₅₋₄=10.0 Hz, ³J₅₋₆=5.0 Hz, ³J₅₋₆=2.5 Hz, H-5), 2.64 (2H, t, ³J_{a-b}=6 Hz, H-b) ppm.

¹³C{¹H} NMR data: (62.9 MHz, CD₃OD, 25° C.) δ=174.2 (C-i); 101.3 (C-1); 74.4 (C-5); 72.4 (C-3); 71.8 (C-a); 68.2 (C-2); 64.3 (C-4); 62.5 (C-6); 51.4 (C-OMe); 35.4 (C-b) ppm.

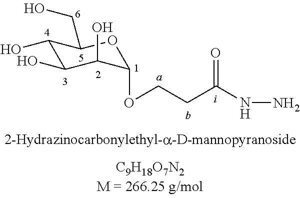 [SMILES: NNC(=O)CCOC1OC(CO)C(O)C(O)C1O]

Aspect: colorless oil

Properties: soluble in H₂O, MeOH, DMF

¹H NMR data: (250 MHz, CD₃OD, 25° C.) δ=4.74 (1H, d, ³J₁₋₂=1.5 Hz, H-1), 3.98 (1H, td, ²Jₐ₋ₐ=10 Hz, ³J_{a-b}=6 Hz, H-a), 3.83 (1H, dd, ²J₆₋₆=11.5 Hz, ³J₅₋₆=2.5 Hz, H-6), 3.74 (1H, dd, ³J₂₋₃=2.5 Hz, ³J₂₋₁=1.5 Hz, H-2), 3.69 (1H, dd, ²J₆₋₆=11.5 Hz, ³J₅₋₆=5.5 Hz, H-6), 3.69 (1H, td, ²Jₐ₋ₐ=10 Hz, ³J_{a-b}=6 Hz, H-a), 3.62-3.55 (2H, m, H-3, H-4), 3.5 (1H, m, ³J₄₋₅=10.0 Hz, ³J₅₋₆=5.5 Hz, ³J₅₋₆=2.5 Hz, H-5), 2.42 (2H, t, ³J_{a-b}=6 Hz, H-b) ppm.

¹³C{¹H} NMR data: (62.9 MHz, CD₃OD, 25° C.) δ=172.9 (C-i); 101.3 (C-1); 74.4 (C-5); 72.4 (C-3); 71.8 (C-a); 68.2 (C-2); 64.3 (C-4); 62.5 (C-6); 35.4 (C-b) ppm.

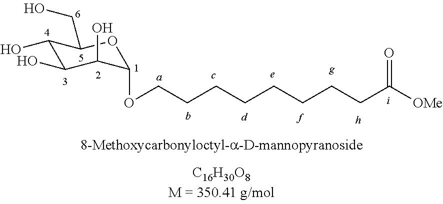 [SMILES: COC(=O)CCCCCCCCOC1OC(CO)C(O)C(O)C1O]

[α]_{D}²⁵=+91 (c=0.16, MeOH)

Aspect: colorless oil

¹H NMR data: (300 MHz, CDCl₃, 25° C.): δ=4.76 (1H, d, ³J₁₋₂=1.5 Hz, H-1), 3.81 (1H, dd, ³J₂₋₃=4.0 Hz, ³J₁₋₂=1.5 Hz, H-2), 3.80-3.65 (4H, m, H-3, H-6, H-6 and H-a), 3.66 (3H, s, H-OMe), 3.65 (1H, t, ³J₃₋₄=9.5 Hz, ³J₅=9.5 Hz, H-4), 3.53 (1H, m, H-5), 3.42 (1H, td, ²Jₐ₋ₐ=9.5 Hz, ³J_{a-b}=6.0 Hz, H-a), 2.32 (2H, t, ³J_{b-9}=7.5 Hz, H-h), 1.60 (4H, m, H-b and H-g), 1.34 (8H, m, H-c, H-d, H-e and H-f) ppm.

¹³C {¹H} NMR data: (75.5 MHz, D₂O, 25° C.): δ=174.6 (C-i); 100.1 (C-1); 73.1 (C-5); 71.3 (C-3); 70.8 (C-2); 67.2 (C-4 and C-a); 61.5 (C-6); 50.7 (C-OMe); 33.4 (C-h); 29.2, 29.0, 28.9, 28.7, 25.9, 24.6 (C-b/c/d/e/f/g) ppm.

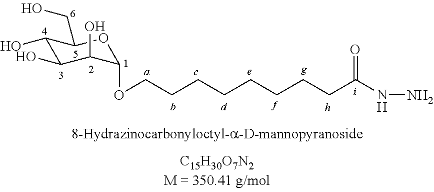 [SMILES: NNC(=O)CCCCCCCCOC1OC(CO)C(O)C(O)C1O]

[α]_{D}²⁵=+49 (c=1.0, MeOH)

Aspect: white powder

¹H NMR data: (300 MHz, CDCl₃, 25° C.): δ=4.76 (1H, d, ³J₁₋₂=1.5 Hz, H-1), 3.81 (1H, dd, ³J₂₋₃=4.0 Hz, ³J₁₋₂=1.5 Hz, H-2), 3.77-3.68 (4H, m, H-3, H-6, H-6 and H-a), 3.64 (1H, t, ³J₃₋₄=9.5 Hz, ³J₅=9.5 Hz, H-4), 3.53 (1H, m, H-5), 3.42 (1H, td, ²Jₐ₋ₐ=9.5 Hz, ³J_{a-b}=6.0 Hz, H-a), 2.32 (2H, t, ³Jₕ₋₉=7.5 Hz, H-h), 1.60 (4H, m, H-b and H-g), 1.34 (8H, m, H-c, H-d, H-e and H-f) ppm.

¹³C {¹H} NMR data: (75.5 MHz, D₂O, 25° C.): δ=173.9 (C-i); 100.2 (C-1); 73.2 (C-5); 71.3 (C-3); 70.8 (C-2); 67.1 (C-4 and C-a); 61.4 (C-6); 33.6 (C-h); 29.2, 28.9 (2), 28.7, 25.8, 25.4 (C-b/c/d/e/f/g) ppm.

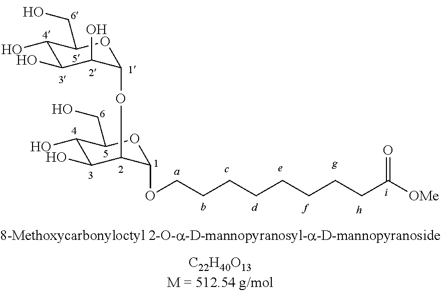 [SMILES: COC(=O)CCCCCCCCOC1OC(CO)C(O)C(O)C1OC1OC(CO)C(O)C(O)C1O]

[α]_{D}²⁵=+64 (c=1.0, MeOH)

Aspect: colorless oil

¹H NMR data: (300 MHz, CD₃OD, 25° C.): δ=5.06 (1H, s, H-1); 4.98 (1H, d, ³J₁₋₂=1.0 Hz, H-1′); 4.00 (1H, m, H-2′); 3.87-3.65 (6H, m, H-2, H-3, H-3′, H-5′ H-6, H-6′ and H-a); 3.67 (3H, s, H-OMe); 3.52 (1H, m, H-5); 3.60 (2H, m, H-4′ and H-4); 3.44 (1H, td, ²Jₐ₋ₐ=9.5 Hz, ³J_{a-b}=6.0 Hz, H-a); 2.33 (2H, t, ³J_{h-g}=7.5 Hz, H-h); 1.30 (4H, m, H-b and H-g); 1.34 (8H, m, H-c, H-d, H-e and H-f) ppm.

¹³C {¹H} NMR data: (75.5 MHz, CD₃OD, 25° C.): δ=174.7 (C-i); 102.8 (C-1′); 98.5 (C-1); 79.3 (C-2); 73.6 (C-5′); 73.2 (C-5); 71.0 and 70.8 (C-3 and C-3′); 70.5 (C-2′); 67.6 (C-a); 67.4 and 67.3 (C-4 and C-4′); 61.7 and 60.6 (C-6 and C-6′); 50.7 (C-OMe); 33.4 (C-h); 29.2, 29.0, 28.9, 28.7, 25.9, 24.6 (C-b/c/d/e/f/g) ppm.

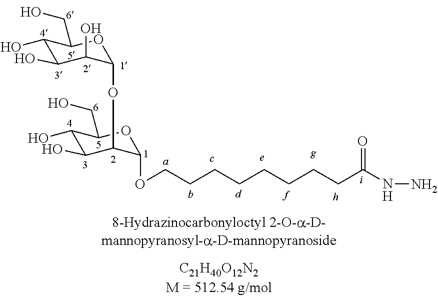 [SMILES: NNC(=O)CCCCCCCCOC1OC(CO)C(O)C(O)C1OC1OC(CO)C(O)C(O)C1O]

[α]_{D}²⁵=+59 (c=1.0, MeOH)

Aspect: colorless oil

Properties: soluble in H₂O, MeOH, DMF

¹H NMR data: (500 MHz, D₂O, 25° C.): δ=5.02 (1H, d, ³J₁₋₂=2.0 Hz, H-1); 4.95 (1H, d, ³J_{1′-2′}=1.5 Hz, H-1′); 4.00 (1H, dd, ³J_{1′-2′}=1.5 Hz, ³J_{2′-3′}=3.0 Hz, H-2′); 3.87 (1H, m, ³J₁₋₂=2.0 Hz, H-2); 3.82 (1H, m, H-3); 3.81 (2H, m, H-6′); 3.77 (1H, dd, ³J_{3′-4′}=10.0 Hz, ³J_{2′-3′}=3.0 Hz, H-3′); 3.68 (1H, m, H-6); 3.67 (1H, m, H-5′); 3.63 (1H, m, ²Jₐ₋ₐ=9.5 Hz, H-a); 3.61 (1H, dd, ³J₃₋₄=9.5 Hz, ³J₄₋₅=9.5 Hz, H-4); 3.56 (1H, m, H-5); 3.55 (1H, t, ³J_{3′-4′}=10.0 Hz, ³J_{4′-5′}=10.0 Hz, H-4′); 3.47 (1H, dt, ²Jₐ₋ₐ=9.5 Hz, ³J_{a-b}=6.0 Hz, H-a); 2.13 (2H, t, ³J_{h-g}=7.5 Hz, H-h); 1.51 (4H, m, H-b and H-g); 1.23 (8H, m, H-c, H-d, H-e and H-f) ppm.

¹³C {¹H} NMR data: (125.8 MHz, D₂O, 25° C.): δ=175.9 (C-i); 102.2 (C-1′); 97.9 (C-1); 78.6 (C-2); 73.2 (C-5′); 72.6 (C-5); 70.2 and 70.1 (C-3 and C-3′); 69.8 (C-2′); 67.8 (C-a); 66.8 and 66.7 (C-4 and C-4′); 60.9 and 60.7 (C-6 and C-6′); 33.5 (C-h); 28.3, 28.1, 28.0, 27.9, 25.1, 24.9 (C-b/c/d/e/f/g) ppm.

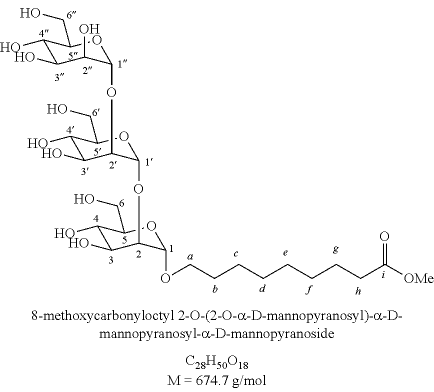 [SMILES: COC(=O)CCCCCCCCOC1OC(CO)C(O)C(O)C1OC1OC(CO)C(O)C(O)C1OC1OC(CO)C(O)C(O)C1O]

[α]_{D}²⁵=+70 (c=1.0, MeOH)

Aspect: colorless oil

¹H NMR data: (500 MHz, CDCl₃, 25° C.): δ=5.28 (1H, d, ³J_{1′-2′}=1.5 Hz, H-1′); 5.07 (1H, d, H-1); 5.00 (1H, d, ³J_{1″-2″}=1.5 Hz, H-1″); 4.06 (1H, dd, ³J_{3′-2′}=3.5 Hz, ³J_{1′-2′}=1.5 Hz, H-2′); 3.99 (1H, dd, ³J_{3″-2″}=3.5 Hz, ³J_{1″-2″}=1.5 Hz, H-2″); 3.91 á 3.80 (5H, m, H-2, H-3′, H-6, H-6′, H-6″); 3.78 á 3.54 (14H, m, H-3, H-3″, H-4, H-4′, H-4″, H-5′, H-5″, H-6, H-6′, H-6″, H-a and H-OMe); 3.52 (1H, m, H-5); 3.44 (1H, td, ²Jₐ₋ₐ=9.5 Hz, ³J_{a-b}=6.5 Hz, H-a); 2.34 (2H, t, ³J_{h-g}=7.5 Hz, H-h); 1.66-1.55 (4H, m, H-b and H-g); 1.43-1.30 (8H, m, H-c, H-d, H-e and H-f) ppm.

¹³C {¹H} NMR data: (125.8 MHz, D₂O, 25° C.): δ=174.5 (C-i); 102.6 (C-1″); 101.0 (C-1′); 98.4 (C-1); 79.4 (C-2); 78.8 (C-5″); 73.4 (C-4″ and C-5′); 73.1 (C-5); 70.9 (C-2″); 70.7 (C-3); 70.4 (C-2′ and C-4′/4); 67.7 (C-3′); 67.5 (C-4/4′); 67.3 (C-3″); 67.1 (C-a); 61.8, 61.7 and 61.5 (3C, C-6/6′/6″); 50.5 (C-OMe); 33.3 (C-h); 29.1, 28.9, 28.8, 28.6, 25.8, 24.5 (C-b/c/d/e/f/g) ppm.

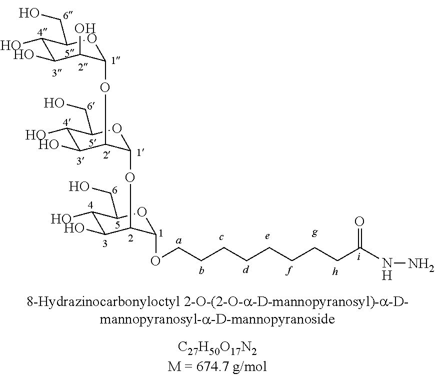 [SMILES: NNC(=O)CCCCCCCCOC1OC(CO)C(O)C(O)C1OC1OC(CO)C(O)C(O)C1OC1OC(CO)C(O)C(O)C1O]

[α]_{D}²⁵=+59 (c=1.0, MeOH)

Aspect: colorless oil

Properties: soluble in H₂O, MeOH, DMF

¹H NMR data: (500 MHz, CDCl₃, 25° C.): δ=5.28 (1H, d, ³J_{1′-2′}=1.5 Hz, H-1′); 5.07 (1H, d, H-1); 5.00 (1H, d, ³J_{1″-2″}=1.5 Hz, H-1″); 4.06 (1H, dd, ³J_{3′-2′}=3.5 Hz, ³J_{1′-2′}=1.5 Hz, H-2′); 3.99 (1H, dd, ³J_{3″-2″}=3.5 Hz, ³J_{1″-2″}=1.5 Hz, H-2″); 3.90 á 3.81 (5H, m, H-2, H-3′, H-6, H-6′ and H-6″); 3.76 á 3.56 (14H, m, H-3, H-3″, H-4, H-4′, H-4″, H-5′, H-5″, H-6, H-6′, H-6″, H-a and H-OMe)); 3.52 (1H, m, H-5′); 3.44 (1H, td, ²J_{a-a′}=9.5 Hz, ³J_{a-b}=6.5 Hz, H-a); 2.17 (2H, t, ³J_{b-g}=7.5 Hz, H-h); 1.66-1.55 (4H, m, H-b and H-g); 1.42-1.32 (8H, m, H-c, H-d, H-e and H-f) ppm.

¹³C {¹H} NMR data: (125.8 MHz, D₂O, 25° C.): δ=173.8 (C-i); 102.6 (C-1″); 101.0 (C-1′); 98.4 (C-1); 79.3 (C-2); 78.7 (C-5″); 73.5 (C-4″ and C-5′); 73.1 (C-5); 70.9 (C-2″); 70.7 (C-3); 70.4 and 70.3 (C-2′ and C-4′/4); 67.6 (C-3′); 67.4 (C-4/4′); 67.2 (C-3″); 67.1 (C-a); 61.6, 61.5 and 61.4 (C-6, C-6′, C-6″); 50.5 (C-OMe); 33.5 (C-h); 29.0, 28.8, 28.8, 28.6, 25.7, 25.2 (C-b/c/d/e/f/g) ppm.

The synthesis route for mannosylated dendrimers as described above allowed the following molecules to be obtained:

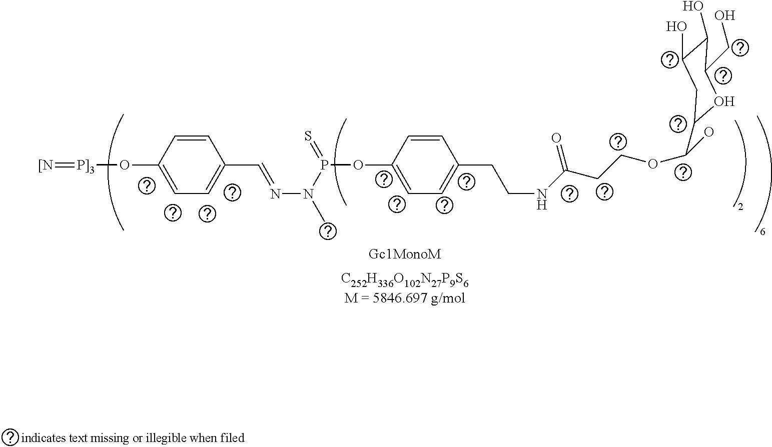

Aspect: yellow powder

¹H NMR data: (500 MHz, D₂O/THF-d8 (2:1), 42° C.): δ=7.91 (6H, s, H-i); 7.74 (12H, d, ³J_{H-H}=7.5 Hz, H-C₁³); 7.24 and 7.16 (48H, d, ³J_{H-H}=10 Hz, H-C₁² and H-C₁³); 6.94 (12H, d, ³J_{H-H}=7.5 Hz, H-C₀²); 4.97 (12H, d, H-1); 4.06 (12H, m, H-a); 3.99 (12H, dd, ²J₆₋₆=12.5 Hz, ³J₆₋₅=4.0 Hz, H-6); 3.92-3.79 (60H, m, H-a, H-2, H-3, H-4 and H-6); 3.71 (12H, m, H-5); 3.44 (24H, t, CH₂—CH₂—NH); 3.33 (18H, d, ³J_{H-P}=10.0 Hz, H-m); 2.83 (24H, t, CH₂—CH₂—NH); 2.60 (24H, t, ³J_{b-a}=7.5 Hz, H-b) ppm.

¹³C {¹H} NMR data: (125 MHz, D₂O/THF-d8 (2:1), 42° C.): δ=172.9 (C-c); 151.1 (C-C₀¹); 149.2 (C-C₁¹); 139.3 (C-i); 136.9 (C-C₁⁴); 132.9 (C-C₀⁴); 130.3 (C-C₁²/C₁³); 128.4 (C-C₀³); 121.3 (C-C₀²); 121.1 (C-C₁²/C-C₁³); 100.1 (C-1); 73.1 (C-5); 71.1 (C-3); 70.5 (C-2); 63.8 (C-a); 61.2 (C-6); 40.8 (CH₂—CH₂—NH); 36.3 (C-b); 34.6 (CH₂—CH₂—NH); 32.8 (³J_{C-P}=11.5 Hz, C-m) ppm.

³¹P{¹H} NMR data: (202 MHz, D₂O/THF-d8 (2:1), 42° C.): δ=63.2 (s, P₁=S); 9.3 (s, N₃P₃) ppm.

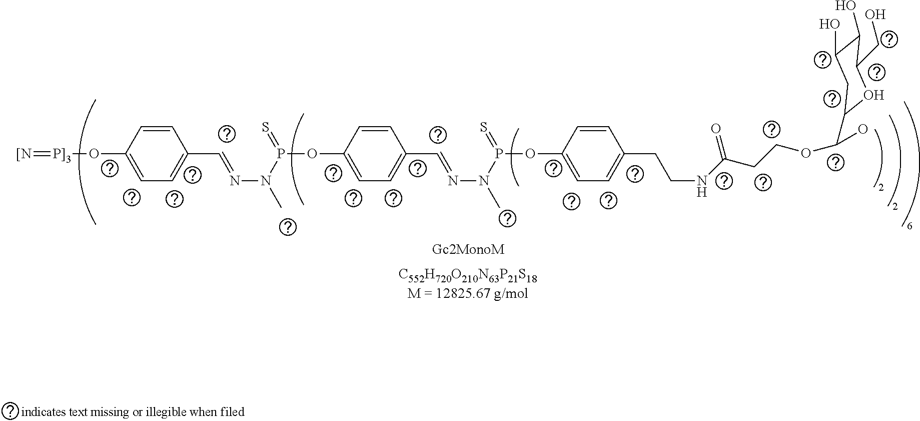

Aspect: yellow powder

¹H NMR data: (500 MHz, D₂O/THF-d8 (2:1), 42° C.): δ=7.94 (18H, broad s, H-i and H-j); 7. 89-6.86 (168H, m, H-C₀², H-C₀³, H-C₁², H-C₁³, H-C₂² and H-C₂³); 4.99 (24H, d, H-1); 4.09 (24H, m, H-a); 3.99 (24H, dd, ²J₆₋₆=13.0 Hz, H-6); 3.94-3.82 (120H, m, H-a, H-2, H-3, H-4 and H-6); 3.75 (24H, m, H-5); 3.48 (48H, broad m, CH₂—CH₂—NH); 3.41 (54H, broad m, H-m and H-n); 2.88 (48H, broad t, CH₂—CH₂—NH); 2.62 (48H, broad t, H-b) ppm.

¹³C NMR data: (125 MHz, D₂O/THF-d8 (2:1), 42° C.): δ=172.8 (C-c); 151.3-149.2 (C-C₀¹, C-C₁¹ and C-C₂¹); 139.8 and 139.4 (C-i and C-j); 139.8-121.2 (C-C₀², C-C₀³, C-C₀⁴, C-C₁², C-C₁³, C-C₁⁴, C-C₂², C-C₂³ and C-C₂⁴); 100.1 (C-1); 73.2 (C-5); 71.2 (C-3); 70.5 (C-2); 63.8 (C-a); 61.3 (C-6); 40.8 (CH₂—CH₂—NH); 36.3 (C-b); 34.6 (CH₂—CH₂—NH); 32.7 (C-m and C-n) ppm.

³¹P{¹H} NMR data: (202 MHz, D₂O/THF-d8 (2:1), 42° C.): δ=63.2 (s, P₂=S); 62.2 (s, P₁=S); 9.2 (s, N₃P₃) ppm.

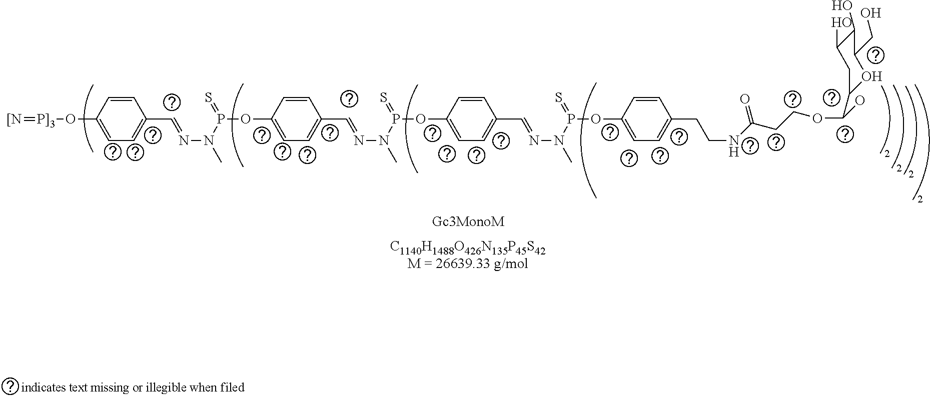

Aspect: yellow powder

¹H NMR data: (500 MHz, D₂O/THF-d8 (1:1), 37° C.): δ=8.09 (42H, broad s, H-i, H-j and H-k); 7.98-7.18 (360H, m, H-C₀², H-C₀³, H-C₁², H-C₁³, H-C₂², H-C₂³, H-C₃² and H-C₃³); 5.06 (48H, d, H-1); 4.17 (48H, m, H-a); 4.06 (48H, dd, ²J₆₋₆=10.0 Hz, H-6); 4.01-3.89 (240H, m, H-a, H-2, H-3, H-4 and H-6); 3.80 (48H, m, H-5); 3.58 (222H, broad m, CH₂—CH₂—NH, H-m, H-n and H-o); 2.99 (96H, broad t, CH₂—CH₂—NH); 2.71 (96H, broad t, H-b) ppm.

¹³C NMR data: (125 MHz, D₂O/THF-d8 (1:1), 37° C.): δ=172.7 (C-c); 151.6-149.3 (C-C₀¹, C-C₁¹, C-C₂¹ and C-C₃¹); 139.6-121.2 (C-i, C-j, C-k, C-C₀², C-C₀³, C-C₀⁴, C-C₁², C-C₁³, C-C₁⁴, C-C₂², C-C₂³ C-C₂⁴ and C-C₃², C-C₃³ C-C₃⁴); 100.2 (C-1); 73.3 (C-5); 71.3 (C-3); 70.6 (C-2); 63.8 (C-a); 61.4 (C-6); 40.9 (CH₂—CH₂—NH); 36.3 (C-b); 34.8 (CH₂—CH₂—NH); 32.8 (C-m C-n and C-o) ppm.

³¹P{¹H} NMR data: (202 MHz, D₂O/THF-d8 (1:1), 37° C.): δ=63.1 (s, P₃═S); 62.5 (s, P₁=S and P₂=S); 9.1 (s, N₃P₃) ppm.

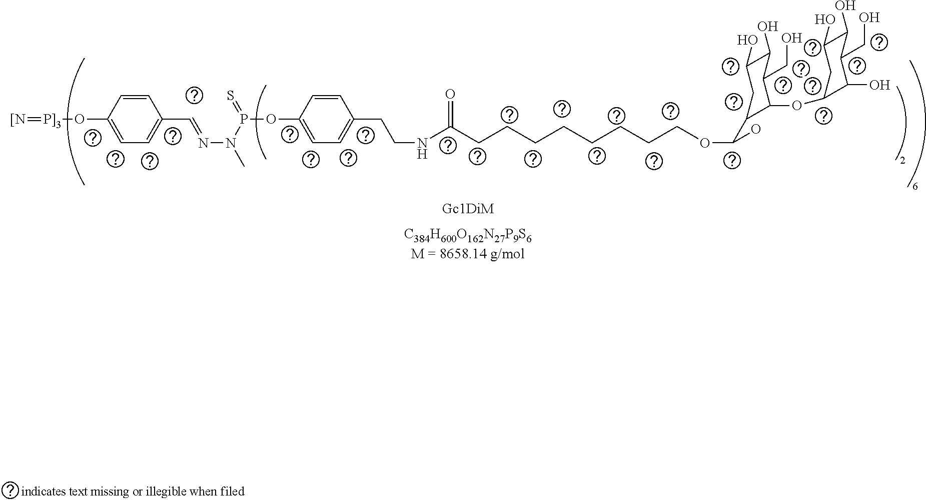

Aspect: yellow powder

¹H NMR data: (500 MHz, D₂O/THF-d8 (2:1), 42° C.): δ=7.88 (6H, s, H-j); 7.73 (12H, d, ³J_{H-H}=7.5 Hz, H-C₀³); 7.20 and 7.15 (48H, d, ³J_{H-H}=10 Hz, H-C₁² and H-C₁³); 6.99 (12H, d, ³J_{H-H}=7.5 Hz, H-C₀²); 5.18 (12H, d, H-1); 5.15 (12H, d, H-1′); 4.22 (12H, dd, H-2′); 4.01-3.78 (108H, m, H-a, H-2, H-3, H-3′, H-4, H-4′, H-5′, H-6 and H-6′); 3.66 (12H, m, H-5); 3.58 (12H, m, H-a) 3.43 (24H, broad t, CH₂—CH₂—NH); 3.31 (18H, d, ³J_{H-P}=8.0 Hz, H-n); 2.82 (24H, broad t, CH₂—CH₂—NH); 2.23 (24H, t, ³J_{h-g}=5.0 Hz, H-h); 1.67 (48H, m, H-b and H-g); 1.38 (96H, m, H-c, H-d, H-e and H-f) ppm.

¹³C {¹H} NMR data: (125 MHz, D₂O/THF-d8 (2:1), 42° C.): δ=174.9 (C-i); 151.2 (C-C₀¹); 149.1 (C-C₁¹); 139.3 (C-j); 136.9 (C-C₁⁴); 132.8 (C-C₀⁴); 130.0 (C-C₁²/C₁³); 128.4 (C-C₀³); 121.1 (C-C₀²); 121.0 (C-C₁²/C-C₁³); 102.7 (C-1′); 98.7 (C-1); 79.2 (C-2); 70.3 (C-2′); 67.9 (C-a); 73.5, 73.2, 70.9, 67.7-66.6, 61.4, 61.2 (C-3, C-3′, C-4, C-4′, C-5, C-5′, C-6 and C-6′); 40.5 (CH₂—CH₂—NH); 36.1 (C-h); 34.7 (CH₂—CH₂—NH); 32.8 (³J_{C-P}=11.5 Hz, C-n); 29.3-25.9 (C-c, C-d, C-e, C-f and C-g) ppm.

³¹P{¹H} NMR data: (202 MHz, D₂O/THF-d8 (2:1), 42° C.): δ=62.9 (s, P₁=S); 9.1 (s, N₃P₃) ppm.

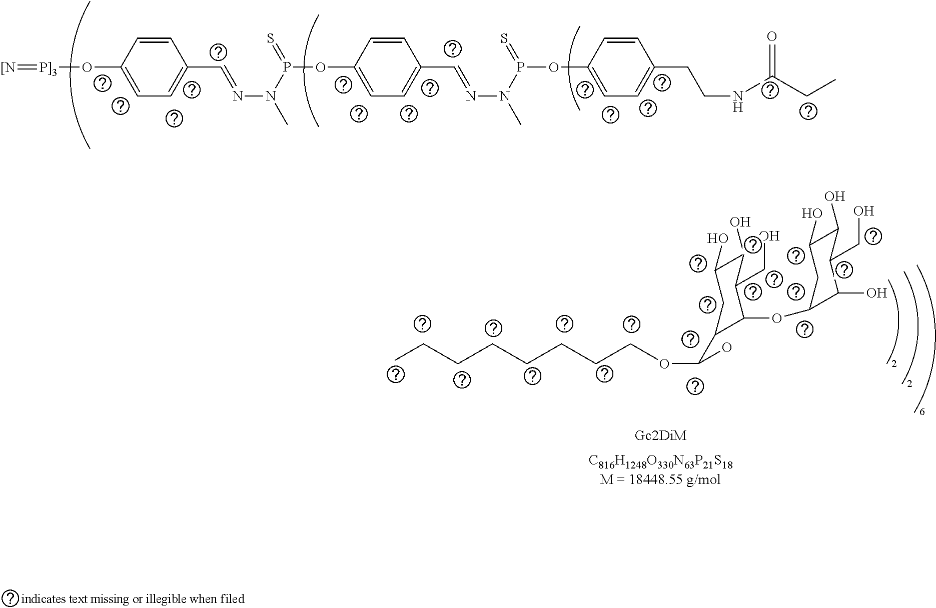

Aspect: yellow powder

¹H NMR data: (500 MHz, D₂O/THF-d8 (2:1), 44° C.): δ=8.10 (18H, broad s, H-j and H-k); 7.97-7.22 (168H, m, H-C₀², H-C₀³, H-C₁², H-C₁³, H-C₂² and H-C₂³); 5.32 (24H, d, H-1); 5.30 (24H, d, H-1′); 4.35 (24H, dd, H-2′); 4.14-3.95 (216H, m, H-a, H-2, H-3, H-3′, H-4, H-4′, H-5′, H-6 and H-6′); 3.80 (24H, m, H-5); 3.72 (24H, m, H-a); 3.61 (102H, broad t, CH₂—CH₂—NH, H-n and H-o); 3.01 (48H, broad t, CH₂—CH₂—NH); 2.40 (24H, broad t, H-h); 1.83 (96H, m, H-b and H-g); 1.56 (192H, m, H-c, H-d, H-e and H-f) ppm.

¹³C {¹H} NMR data: (125 MHz, D₂O/THF-d8 (2:1), 44° C.): δ=174.7 (C-i); 151.5-149.4 (C-C₀¹, C-C₁¹ and C-C₂¹); 139.5 (C-j and C-k); 137.1-121.2 (C-C₀², C-C₀³, C-C₀⁴, C-C₁², C-C₁³, C-C₁⁴, C-C₂², C-C₂³ and C-C₂⁴); 102.9 (C-1′); 98.9 (C-1); 79.3 (C-2); 70.5 (C-2′); 67.9 (C-a); 73.7, 73.4, 71.1, 67.9-66.8, 61.6, 61.4 (C-3, C-3′, C-4, C-4′, C-5, C-5′, C-6 and C-6′); 40.7 (CH₂—CH₂—NH); 36.2 (C-h); 34.9 (CH₂—CH₂—NH); 32.9 (C-n and C-o); 29.5-24.9 (C-c, C-d, C-e, C-f and C-g) ppm.

³¹P{¹H} NMR data: (202 MHz, D₂O/THF-d8 (2:1), 44° C.): δ=63.1 (s, P₂=S); 62.5 (s, P₁=S); 9.0 (s, N₃P₃) ppm.

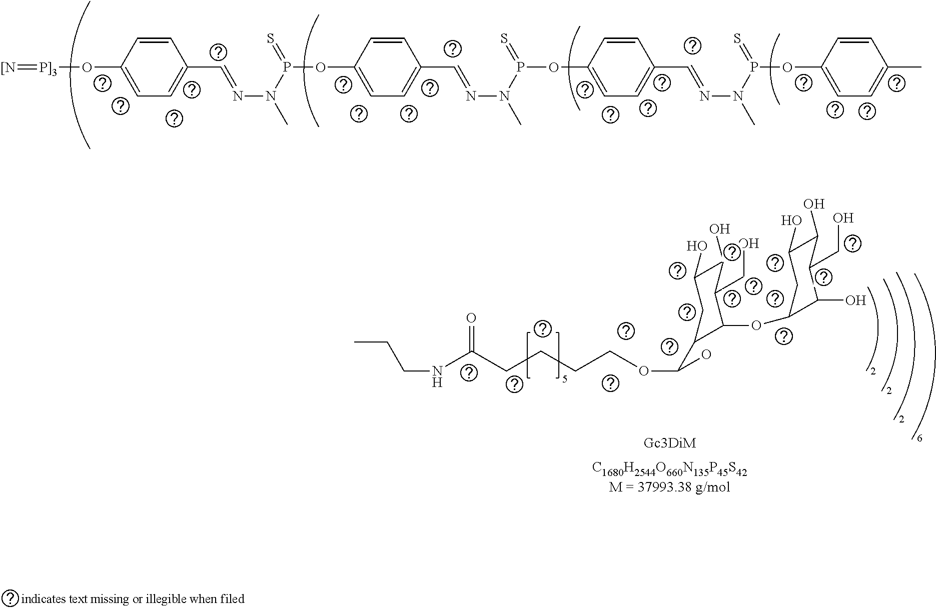

Aspect: yellow powder

¹H NMR data: (500 MHz, D₂O/THF-d8 (2:1), 44° C.): δ=7.84 (48H, broad s, H-j, H-k and H-l); 7.76-7.18 (360H, m, H-C₀², H-C₀³, H-C₁², H-C₁³, H-C₂², H-C₂³, H-C₃² and H-C₃³); 5.19 (48H, d, H-1); 5.17 (48H, d, H-1′); 4.23 (48H, dd, H-2′); 4.02-3.81 (432H, m, H-a, H-2, H-3, H-3′, H-4, H-4′, H-5′, H-6 and H-6′); 3.66 (48H, m, H-5); 3.58 (48H, m, H-a); 3.44-3.32 (222H, broad m, CH₂—CH₂—NH, H-n, H-o and HID); 2.82 (48H, broad m, CH₂—CH₂—NH); 2.23 (48H, broad t, H-h); 1.69-1.65 (192H, broad m, H-b and H-g); 1.39 (384H, broad m, H-c, H-d, H-e and H-f) ppm.

¹³C {¹H} NMR data: (125 MHz, D₂O/THF-d8 (2:1), 44° C.): δ=174.7 (C-i); 149.2 (C-C₀¹, C-C₁¹, C-C₂¹ and C-C₃¹); 136.8-121.1 (C-j, C-k, C-l, C-C₀², C-C₀³, C-C₀⁴, C-C₁², C-C₁³, C-C₁⁴, C-C₂², C-C₂³ C-C₂⁴ and C-C₃², C-C₃³ C-C₃⁴), 102.7 (C-1′); 98.7 (C-1); 79.2 (C-2); 70.4 (C-2′); 67.9 (C-a); 73.6, 73.2, 70.9, 67.9, 67.7, 67.1, 61.4, 61.2 (C-3, C-3′, C-4, C-4′, C-5, C-5′, C-6 and C-6′); 40.5 (CH₂—CH₂—NH); 36.2 (C-h); 34.7 (CH₂—CH₂—NH); 32.8 (C-n C-o and C-p); 29.4-25.9 (C-c, C-d, C-e, C-f and C-g) ppm.

³¹P{¹H} NMR data: (202 MHz, D₂O/THF-d8 (2:1), 44° C.): δ=62.9 (s, P₃=S); 62.0 (s, P₁=S and P₂=S); 9.0 (s, N₃P₃) ppm.

[symbol]

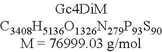

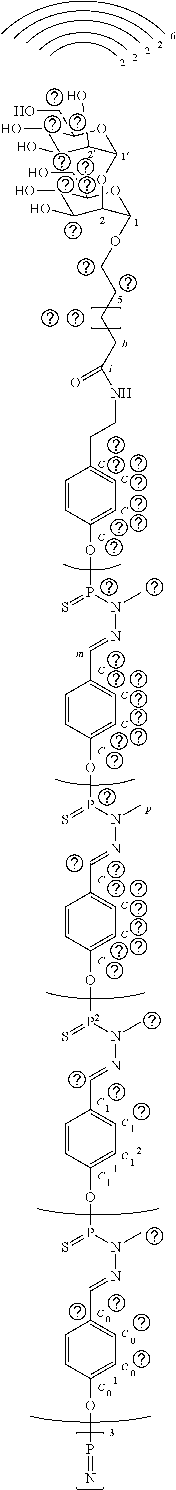

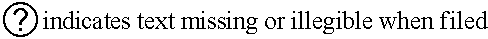

Aspect: yellow powder

¹H NMR data: (500 MHz, D₂O/THF-d8 (1:1), 44° C.): δ=7.85 (96H, broad s, H-j, H-k, H-l and H-m); 7.80-7.22 (744H, m, H-C₀², H-C₀³, H-C₁², H-C₁³, H-C₂², H-C₂³, H-C₃², H-C₃³, H-C₄² and H-C₄³); 5.17 (96H, d, H-1); 5.14 (96H, d, H-1′); 4.18 (96H, dd, H-2′); 4.01-3.85 (864H, m, H-a, H-2, H-3, H-3′, H-4, H-4′, H-5′, H-6 and H-6′); 3.66 (96H, m, H-5); 3.56 (96H, m, H-a); 3.45-3.33 (462H, broad m, CH₂—CH₂—NH, H-n, H-o, H-p and H-q); 2.82 (192H, broad m, CH₂—CH₂—NH); 2.22 (96H, broad t, H-h); 1.70-1.64 (384H, broad m, H-b and H-g); 1.37 (768H, broad m, H-c, H-d, H-e and H-f) ppm.

¹³C {¹H} NMR data: (125 MHz, D₂O/THF-d8 (1:1), 44° C.): δ=174.8 (C-i); 149.3 (C-C₀¹, C-C₁¹, C-C₂¹, C-C₃¹ and C-C₄¹); 136.7-120.9 (C-j, C-k, C-l, C-m, C-C₀², C-C₀³, C-C₀⁴, C-C₁², C-C₁³, C-C₁⁴, C-C₂², C-C₂³ C-C₂⁴ and C-C₃², C-C₃³ C-C₃⁴), 102.6 (C-1′); 98.6 (C-1); 79.3 (C-2); 70.6 (C-2′); 67.5 (C-a); 73.8-61.4 (C-3, C-3′, C-4, C-4′, C-5, C-5′, C-6 and C-6′); 40.7 (CH₂—CH₂—NH); 36.1 (C-h); 34.8 (CH₂—CH₂—NH); 32.6 (C-n C-o, C-p and C-q); 29.6-25.8 (C-c, C-d, C-e, C-f and C-g) ppm.

³¹P{¹H} NMR data: (202 MHz, D₂O/THF-d8 (1:1), 44° C.): δ=63.1 (s, P₄=S); 62.6 (broad s, P₁=S, P₂=S and P₃=S); 9.1 (s, N₃P₃) ppm.

[symbol]

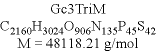

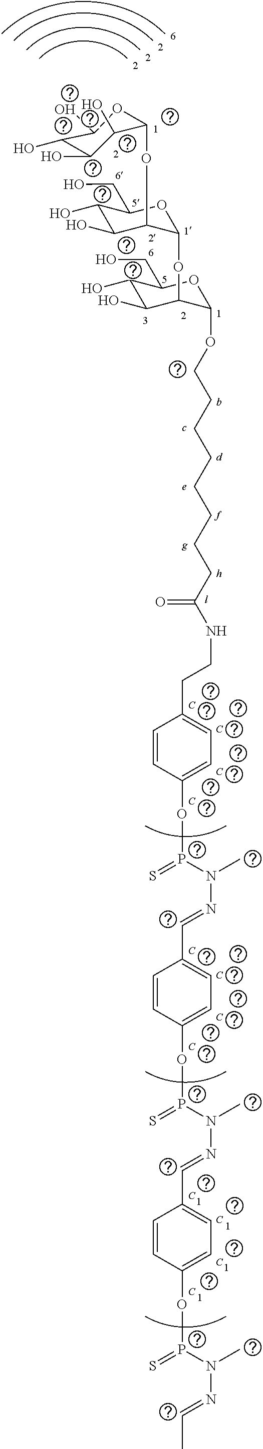

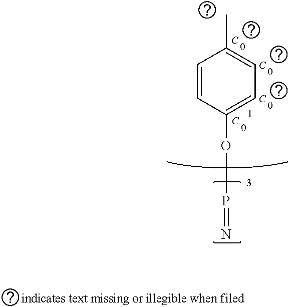

Aspect: yellow powder

¹H NMR data: (500 MHz, D₂O/THF-d8 (1:1), 44° C.): δ=8.22 (48H, broad s, H-j, H-k and H-l); 7.97-7.26 (360H, m, H-C₀³, H-C₁², H-C₁³, H-C₂², H-C₂³, H-C₃² and H-C₃³); 5.48 (48H, d, H-1′); 5.28 (48H, d, H-1); 5.22 (48H, d, H-1″); 4.33 and 4.28 (48H, broad dd, H-2′ and H-2); 4.16-3.86 (624H, m, H-a, H-2″, H-3, H-3′, H-3″, H-4, H-4′, H-4″, H-5′, H-5″, H-6, H-6′ and H-6″); 3.71 (48H, m, H-5); 3.65 (48H, m, H-a); 3.52 (222H, broad m, CH₂—CH₂—NH, H-n, H-o and H-p); 2.92 (48H, broad m, CH₂—CH₂—NH); 2.31 (48H, broad t, H-h); 1.73 (192H, broad m, H-b and H-g); 1.45 (384H, broad m, H-c, H-d, H-e and H-f) ppm.

¹³C {¹H} NMR data: (125 MHz, D₂O/THF-d8 (1:1), 44° C.): δ=174.1 (C-i); 149.4 (C-C₀¹, C-C₁¹, C-C₂¹ and C-C₃¹); 136.9-121.1 (C-j, C-k, C-l, C-C₀², C-C₀³, C-C₀⁴, C-C₁², C-C₁³, C-C₁⁴, C-C₂², C-C₂³ C-C₂⁴ and C-C₃², C-C₃³ C-C₃⁴), 102.55 (C-1); 101.2 (C-1′); 98.8 (C-1″); 78.7 (C-2′); 70.4 (C-2); 67.9 (C-a); 73.6, 73.3, 71.5, 70.9, 70.6, 67.7-66.8, 61.5, 61.3 (C-2″, C-3, C-3′, C-3″, C-4, C-4′, C-4″, C-5, C-5′, C-5″, C-6, C-6′ and C-6″); 40.6 (CH₂—CH₂—NH); 36.2 (C-h); 34.8 (CH₂—CH₂—NH); 32.8 (C-n C-o and C-p); 29.4-24.1 (C-c, C-d, C-e, C-f and C-g) ppm.

³¹P{¹H} NMR data: (202 MHz, D₂O/THF-d8 (1:1), 44° C.): δ=63.0 (s, P₃=S); 62.2 (s, P₁=S and P₂═S); 9.2 (s, N₃P₃) ppm.

III. Demonstrating the Anti-Inflammatory Properties of Mannodendrimers in Vitro

1. Inhibition of the Binding of HEK Cells: DC-SIGN to Mannan (Borrok, M. Et al. J Am Chem Soc 129, 12780-12785 (2007))

50 μL of mannan from Saccharomyces cerevisiae at 200 μg/mL in solution in an ethanol/water mixture (1:1) are deposited in microplate wells and the solvent is then evaporated. The wells are saturated for 2 hours at room temperature with 200 μL of PBS CaCl₂ 5% BSA. During this time, transfected HEK cells for the DC-SIGN receptor merged with Green Fluorescent Protein (GFP) and with DsRed are marked with succinimidyl ester of carboxyfluorescein di-acetate (CFDA, SE) at 10 μM in 2 mL of PBS, for 5 minutes at 37° C. The marking is stopped by adding 12 mL of PBS CaCl₂ 0.5% BSA (Buffer A). After centrifugation, the cells are taken up in an amount of 0.8.10⁶ cells/mL in Buffer A. The inhibition takes place with co-incubation of 50 μL of Buffer A or of a mannodendrimer solution at different concentrations and 50 μL of a cell suspension (40,000 cells) in each well for 1 hour at 37° C. away from light. The interaction of the DC-SIGN receptor expressed by the cells with mannan allows retention of the cells in the wells. The suspended cells bound to the mannodendrimer are delicately removed with two washings with Buffer A. The adherent cells are then lyzed by adding 100 μL per well of 25 mM Tris buffer, pH 8.4, 0.1% SDS. The fluorescence intensity of each well is quantified with a spectrofluorimeter at λₑₘ=530 nm (λₑₓ=488 nm). The calculation of the percentages and the plotting of the inhibition curves allowing determination of the concentration for which 50% of the effect (EC50) are observed, is performed by means of the software package GraphPad Prism.

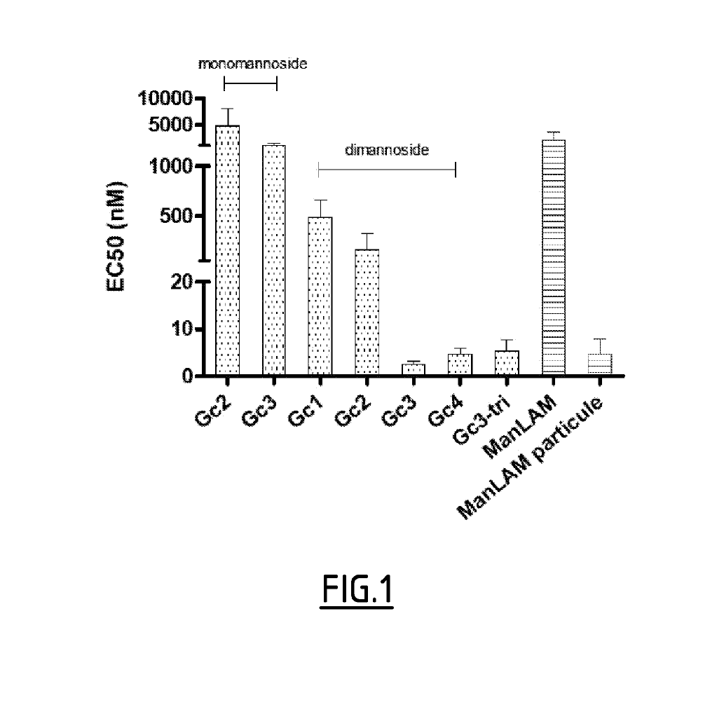

This procedure is applied with dendrimers prepared according to the invention. The results are gathered in FIG. 1.

2. Inhibition of the Production of Cytokine by Dendritic Cells

a) Purification of Monocytes Stemming from the Blood of a Healthy Donor

The mononucleated cells of circulating blood (PBMC) are obtained from a buffy coat delivered by the Etablissement Francais du Sang (EFS). They are isolated by means of an MSL density gradient (Eurobio) by centrifugation (300 g, 30 mins at room temperature). After three steps of deplating (taking up the pellet in PBS, centrifugation at 120 g, 10 mins at room temperature), the monocytes are purified by positive sorting out on a column by means of magnetic beads coupled with an anti-CD14 antibody (MACS system, CD14 Microbeads, human, 130-050-201, Miltenyi Biotec, Auburn, Calif., USA). The cells are incubated with the beads for 30 minutes at 4° C., with stirring. After washing in decomplemented PBS 0.5% FCS (Fetal Calf Serum) (Lonza Bioscience) 2 mM EDTA (Sigma), the beads/cells suspension is deposited on a column placed in front of a magnet (LS, Miltenyi Biotech). After 3 washings, the column is removed from the magnet and the positive CD14 cells are eluted. The obtained purity level at the end of the column is checked by flow cytometry (>96%). The monocytes are then sown in a 6-well plate, at a concentration of 3.10⁶ cells/well (2 mL) in RPMI Ultraglutamine, 10% decomplemented FCS, 5 mM of β-mercaptoethanol (Sigma), 5.10⁵ U/mL of GM-CSF and 1.10⁵ U/mL of IL-4, for 5 days of differentiation. Every two days, 500 μL/well of RPMI 10% decomplemented FCS, 50 μM β-mercaptoethanol medium are added containing the total dose of cytokines for the volume of each well.

b) Activation of Dendritic Cells

Immature dendritic cells on the 5^{th} or 6^{th} day of cultivation are stimulated for 18 hours with 20 ng/mL of ultra pure LPS from E. coli (K12 fraction, Invivogen) in co-incubation with 10 μg/mL of ManLAM bovis BCG (0.6 μM) or 0.3 and 0.6 μM of mannodendrimers prepared according to the invention or of the culture medium (RPMI Ultraglutamine, 10% decomplemented FCS, 50 μM β-mercaptoethanol) in a final volume of 200 μL, in a microplate.

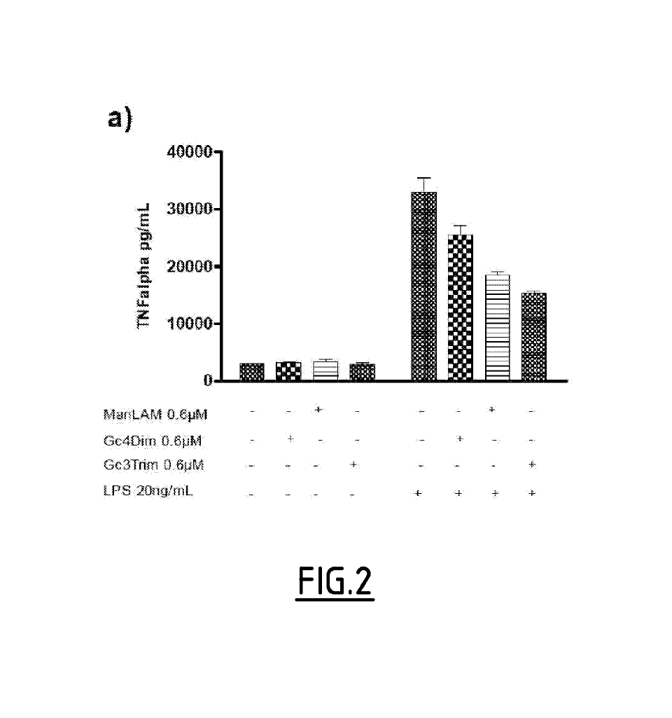

The results of these experiments are illustrated in FIG. 2.

The effect of ManLAM or of the mannodendrimers prepared according to the invention on the activation of dendritic cells by LPS via DC-SIGN is confirmed by pre-incubation (30 mins at 37° C.) of immature cells with an anti-DC-SIGN antagonistic antibody dialyzed beforehand (AZN-D1, 20 μg/mL, Beckman Coulter). The cell culture medium is harvested after 18 hours and is kept at −20° C. The determination of the amount of cytokines (TNF-α) produced and secreted by the cells is measured by means of an ELISA kit (Diaclone), according to the instructions of the manufacturer.

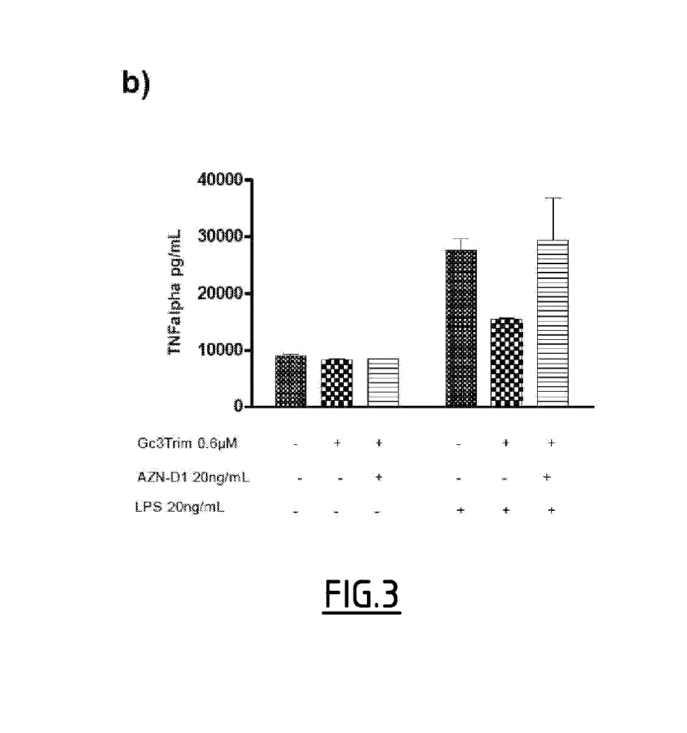

The results of these experiments are illustrated in FIG. 3.

IV. Demonstrating the Anti-Inflammatory Properties of the Mannodendrimers In Vivo

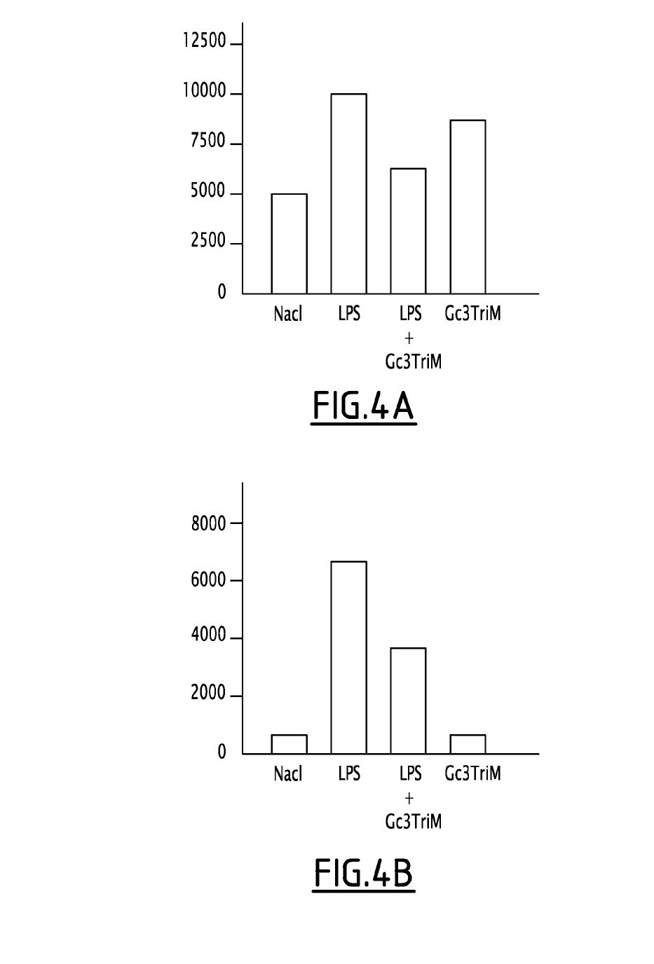

Mice with a genetic background Balb/c were force-fed for 15 days with a dose of mannodendrimer (1 mg/kg/day) and the animals were then subject to nebulization of bacterial lipopolysaccharide (LPS from Pseudomonas aeruginosa, for 30 minutes, 1 mg LPS/kg of mouse) so as to trigger a lung inflammation. After 24 hours, the mice were subject to broncho-alveolar washing and the recovered cells are identified and counted. The results of these experiments are illustrated in FIGS. 4A and 4B.

Generally, the stimulation by LPS induces at the lungs a recruitment of leukocyte cells, mainly macrophages, neutrophilic polynuclear cells and to a lesser extent eosinophilic cells (cf. FIGS. 4A and 4B, comparison of the saline NaCl control with LPS).

It is noted that the mannodendrimer Gc3TriM preventively inhibits recruitment of leukocyte cells in the lungs stimulated with LPS (FIG. 4A, LPS/LPS+Gc3TriM), and that this inhibiting effect is also observed on the recruitment of neutrophils (FIG. 4B, LPS/LPS+Gc3TriM).

It is also noted that Gc3TriM alone induces cell recruitment in the lungs (FIG. 4A NaCl/Gc3TriM control), but that the recruited cells are essentially macrophages, the level of neutrophils not being significantly different from that of the control (FIG. 4B, NaCl/Gc3TriM control).

Histological sections of mice lungs, after broncho-alveolar washing were made and stained with hematoxylin and eosin. These sections show thickening of the bronchial epithelium, a sign of an inflammation in the case of treatment with LPS. This thickening is clearly less in the case of treatment with LPS+Gc3TriM which confirms control of the inflammatory process by the mannodendrimer. Finally, the histological sections in the case of the treatment with Gc3TriM alone do not show any difference with the saline control, the observed recruitment of leukocyte cells therefore does not correspond to an inflammatory process of the epithelium, but to a recruitment of non-activated macrophages.

1-17. (canceled)

18. A dendrimer of generation g comprising:
a central core Φ of valency v;
generation chains with a tree-structure around the core;
an intermediate chain at the end of each chain of generation g or at the end of each bond around the core when g=0; and
a terminal group Σ at the end of each intermediate chain,
characterized in that each Σ group, either identical or different represents independently a monosaccharide, oligosaccharide or polysaccharide group, consisting of a saccharide units, and wherein:
g is an integer comprised between 0 and 10,
v is an integer comprised between 1 and 10,
σ is an integer comprised between 1 and 10,
said intermediate chain is represented by the formula (CI):
-A-(C═O)—Y—CH₂—O— (CI)
wherein:
Y represents a C₁-C₂₀ alkyl group, optionally substituted with a group selected from the group consisting of a halogen, OH, O-alkyl, CF₃, aryl, CN;
A represents a bond or a group:
—O—Ar—X—NR₁—
wherein:
Ar represents an aromatic ring optionally substituted with a group selected from the group consisting of: a halogen, OH, O-alkyl, CF₃, aryl, CN,
R₁ represents a hydrogen atom or a C₁-C₆ alkyl group, and
X represents a C₁-C₆ alkyl group, optionally substituted with a group selected from the group consisting of: halogen, OH, O-alkyl, CF₃, aryl, CN.

19. The dendrimer according to claim 18, such that the intermediate chain is represented by formula (CI′):
—O—C₆H₄—CH₂—CH₂—NH—(C═O)—(CH₂)ₙ—CH₂—O— (CI′)
wherein n is an integer comprised between 1 and 12 and —C₆H₄— represents a divalent phenylene group.

20. The dendrimer according to claim 18, such that it has a structure of the PMMH, PMMH, DAB-AM, PAMAM, PPI, polylysine or polytriazine type.

21. The dendrimer according to claim 18, such that the generation chains are represented by the formula (CG):
—O—Ar′—Z═N—NR₂—(P═S)< (CG)
wherein:
Ar′ is defined as Ar,
Z is defined like X,
R₂ is defined like R₁, and
<represents two bonds located on the phosphorus atom.

22. The dendrimer according to claim 18, such that the central core (I) is selected from the following groups:

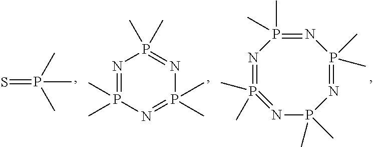

23. The dendrimer according to claim 18, such that g is an integer comprised between 0 and 10.

24. The dendrimer according to claim 18, such that the groups Σ consist of at most 3 saccharide units, either identical or different.

25. The dendrimer according to claim 18, such that the saccharide units forming the Σ groups are selected from the group of hexoses.

26. The dendrimer according to claim 18 such that the Σ groups are identical and consist of mannose.

27. The dendrimer according to claim 18, such that the Σ groups are identical and consist of dimannosides or trimannosides.

28. The dendrimer according to claim 18, such that the Σ groups are identical and consist of glucose.

29. The dendrimer according to claim 18, such that the Σ groups are identical and consist of diglucosides or triglucosides.

30. The dendrimer according to claim 18, such that it is represented according to the following formula (1):
Φ-{{O—C₆H₄—(CH)═N—N(CH₃)—(P═S)<}^{g}[O—C₆H₄—CH₂—CH₂—NH—(C═O)—(CH₂)ₙ—CH₂—O-Σ]₂}ᵥ (1)
wherein:
{ }^{g} designates the tree-structure of the generation chains of said dendrimer.

31. A method for preparing a dendrimer according to claim 18, comprising the reaction of the dendrimer of generation g comprising:
a central core Φ of valency v;
generation chains with a tree-structure around the core:
—NHR₁ terminal groups;
with an acyl-azide compound of formula (3):
N₃—(C═O)—Y—CH₂—O-Σ (3)
wherein: the —NHR₁ groups are possibly in the form of ammonium ions NH₂R₁⁺, in equilibrium with the conjugate base of a weak or strong acid.

32. A method for preparing a dendrimer according to claim 18, comprising the reaction of the dendrimer of formula (2):
Φ—{{O—Ar′—Z═N—NR₂—(P═S)<}^{g}[O—Ar—X—NHR₁]₂}ᵥ (2)
with an acyl-azide compound of formula (3):
N₃—(C═O)—Y—CH₂—O-Σ (3)
wherein: the —NHR₁ groups are possibly in the form of ammonium ions NH₂R₁⁺, in equilibrium with the conjugate base of a weak or strong acid.

33. Compounds of formula (3):
N₃—(C═O)—Y—CH₂—O-Σ (3)
wherein:
σ is an integer comprised between 1 and 10.

34. A method for preparing a compound of formula (3) according to claim 33 comprising:
(i′) the reaction of the ester of formula (7):
R₃O—(C═O)—Y—CH₂—O-Σ (7)
with hydrazine hydrate,
(ii′) followed by the reaction of the compound obtained in (i′) with sodium nitrite in an acid medium,
wherein R₃ represents a linear C₁-C₆ alkyl chain.

35. A drug comprising a dendrimer according to claim 18 and a pharmaceutically acceptable excipient.

36. A method for treating and/or preventing inflammatory disorders comprising administering to a patient in need thereof a drug according to claim 35.
